(19) United States
(12) Patent Application Publication
(10) Pub. No.: US 2019/0254601 A1
(43) Pub. Date: Aug. 22, 2019

(54) BIO-PATCH AND RELATED METHODS FOR DETECTING FERTILITY CONDITION IN COWS

(71) Applicant: LIFE PATCH INTERNATIONAL, Irvine, CA (US)

(72) Inventors: James Christopher Lars BLACKMORE, Los Angeles, CA (US); Donald BOLLELLA, Irvine, CA (US)

(21) Appl. No.: 16/278,145

(22) Filed: Feb. 17, 2019

Related U.S. Application Data
(60) Provisional application No. 62/631,645, filed on Feb. 17, 2018.

(51) Int. Cl. A61B 5/00; A61D 17/00; A01K 29/00

(52) U.S. Cl. CPC A61B 5/6833; G01N 33/74; A01K 29/005; A61D 17/002

(57) ABSTRACT

Bio-patch and related methods for detecting fertility condition in cows. In some embodiments, a patch can include a patch substrate configured to support a plurality of components, and to allow the patch to be attached to a cow. The patch can further include a detector implemented on or at least partially within the patch structure, and configured to detect one or more conditions associated with a fertility cycle of the cow. The patch can further include an interface component implemented on or at least partially within the patch structure, and in communication with the detector. The interface component can be configured to transmit information related to the one or more conditions, such that the information allows estimation of occurrence of a standing estrus window for the cow.

### CROSS-REFERENCE TO RELATED APPLICATION(S)

This application claims priority to U.S. Provisional Application No. 62/631,645 filed Feb. 17, 2018, entitled BIO-PATCH AND RELATED METHODS FOR DETECTING FERTILITY CONDITION IN COWS, the disclosure of which is hereby expressly incorporated by reference herein in its respective entirety.

### BACKGROUND

### Field

The present disclosure relates to a patch configured to detect fertility condition in animals such as cows.

### Description of the Related Art

Some animals such as cows have a relatively small reproductive window of time. Thus, managing cows for producing offsprings and milk can become challenging. Such a challenge can be especially true with a large number of cows grazing in a large area, and with methods such as artificial insemination being costly.

### SUMMARY

In accordance with a number of implementations, the present disclosure relates to a patch that includes a patch substrate configured to support a plurality of components, and to allow the patch to be attached to a cow. The patch further includes a detector implemented on or at least partially within the patch structure, and configured to detect one or more conditions associated with a fertility cycle of the cow. The patch further includes an interface component implemented on or at least partially within the patch structure, and in communication with the detector. The interface component is configured to transmit information related to the one or more conditions, such that the information allows estimation of occurrence of a standing estrus window for the cow.

In some embodiments, the patch substrate can be configured to be attached to a skin of the cow.

In some embodiments, the detector can be configured to detect presence and/or concentration of a hormone associated with the fertility cycle of the cow. For example, the detector can be configured to measure a concentration level of progesterone hormone. The detector can be further configured to generate a signal indicative of an approach of the standing estrus window when the concentration levels measured at two different times indicate a decrease from a first value above a threshold to a second value below the threshold.

In another example, the detector can be configured to measure a concentration level of estradiol hormone. The detector can be further configured to generate a signal indicative of a beginning of the standing estrus window when the concentration levels measured at two different times indicate an increase from a first value below a threshold to a second value above the threshold.

In yet another example, the detector can be configured to measure a concentration level of luteinizing hormone. The detector can be further configured to generate a signal indicative of a beginning of the standing estrus window when the concentration levels measured at two different times indicate an increase from a first value below a threshold to a second value above the threshold.

In some embodiments, the detector can be configured to detect a rate of change in concentration of a hormone associated with the fertility cycle of the cow.

In some embodiments, the detector can be configured to detect an activity of the cow, with the activity being associated with the fertility cycle of the cow. For example, the activity can include an increase in number of steps taken by the cow within a given time interval. For such an activity, the detector can include a pedometer functionality. In another example, the activity can include a mounting action associated with the cow. Such mounting action can be performed by the cow on another cow. For such an activity, the detector can include a tilt sensing functionality. In yet another example, the activity can include the cow being in close proximity of another cow. For such an activity, the detector can include a proximity sensing functionality with respect to a patch attached to the other cow.

In some embodiments, the interface component can include a communication circuit configured to send the information to an external device. The communication circuit can be configured to send the information in a wireless manner.

In some teachings, the present disclosure relates to a method that includes attaching a patch to a cow, with the patch including a substrate configured to support a plurality of components. The method further includes detecting, with the patch, one or more conditions associated with a fertility cycle of the cow. The method further includes transferring information about the detecting such that the information allows estimation of occurrence of a standing estrus window for the cow.

According to some implementations, the present disclosure relates to a system for managing fertility of cows. The system includes a patch configured to be attached to a cow, and including a detector configured to detect one or more conditions associated with a fertility cycle of the cow. The patch further includes an interface component in communication with the detector, and configured to transmit information related to the one or more conditions. The system further includes a monitor configured to receive the information from the patch, such that the information received by the monitor allows estimation of occurrence of a standing estrus window for the cow.

In some embodiments, the monitor can be implemented at a substantially stationary location. In some embodiments, the interface component can be configured to transmit the information to the monitor in a wireless manner.

In some embodiments, the system can further include a relay device implemented to relay the information from the interface component of the patch to the monitor. In some embodiments, the relay device can be implemented on a movable device. For example, the movable device can include a drone capable of flight. the drone can be configured to be capable of flying in a programmed pattern over an area that includes a location of the cow.

In some embodiments, the system can further include a plurality of additional patches, with each being similar to the patch. Each patch can include an identifier such that the patch is associated with the respective cow.

In some implementations, the present disclosure relates to a kit for managing fertility of a cow. The kit includes a patch configured to be attached to a cow. The patch includes a detector configured to detect one or more conditions associated with a fertility cycle of the cow, and an interface component in communication with the detector and configured to transmit information related to the one or more conditions. The kit further includes a printed instruction configured to facilitate use of the patch.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 depicts a bio-patch having one or more features as described herein, being utilized for monitoring one or more biological conditions of an animal.

FIG. 2 shows a process that can be implemented with a patch having one or more features as described herein.

FIG. 3A shows a more specific example of FIG. 2 where a patch can be configured to indicate a time-sensitive condition such as a fertility condition in a cow based on an analysis of a biological fluid.

FIG. 3B shows another more specific example of FIG. 2 where a patch can be configured to indicate a time-sensitive condition such as a fertility condition in a cow based on measurement of a physical/behavioral activity such as walking activity.

FIG. 3C shows yet another more specific example of FIG. 2 where a patch can be configured to indicate a time-sensitive condition such as a fertility condition in a cow based on detection of a physical/behavioral activity such as mounting activity.

FIG. 3D shows yet another more specific example of FIG. 2 where a patch can be configured to indicate a time-sensitive condition such as a fertility condition in a cow based on detection of a physical/behavioral activity such as close contact with other cow(s).

FIG. 4 depicts a cow that can be a more specific example of the animal of FIG. 1.

FIG. 5 shows that in some embodiments, the bio-patch of FIG. 1 can include a number of components.

FIGS. 6A and 6B show a more specific example of the bio-patch of FIG. 5, in which the components can include a bovine fertility assay component.

FIG. 7 shows another more specific example of the bio-patch of FIG. 5, in which the components can include one or more physical activity sensors.

FIG. 8 shows yet another more specific example of the bio-patch of FIG. 5, in which the components can include a proximity sensor to detect a behavioral activity of a cow.

FIG. 9 shows plots of time-dependent concentration levels of example hormones present in cows.

FIG. 10 shows examples of time periods at or about the standing estrus window of FIG. 9.

FIG. 11 shows examples of samplings that can be performed at different times in the example fertility cycle of FIG. 9.

FIG. 12 shows an example measurement situation where two measurements can be made to determine a change in concentration level of a hormone.

FIG. 13 shows another example measurement situation where two measurements can be made to determine a change in concentration level of a hormone.

FIG. 14 shows an example sampling configuration where P4 hormone can be sampled with different frequencies, depending on detection of an increase and a decrease in the P4 level.

FIG. 15 shows a plot of E2 hormone, where the concentration level peaks at or near the estrus window.

FIG. 16 shows that in some embodiments, a patch having one or more features as described herein can include a transmission functionality.

FIG. 17 shows that in some embodiments, a patch having one or more features as described herein can include transmit and receive functionalities.

FIG. 18 shows that a monitor in communication with a patch can include an electronic device having a receiver functionality and a processor.

FIG. 19 shows that a monitor in communication with a patch through a relay can include an electronic device having a receiver functionality and a processor.

FIG. 20 shows a more specific example of the relay-facilitated communication configuration of FIG. 19.

FIG. 21 shows an example of how the relay-facilitated communication configuration of FIG. 20 can be implemented.

FIG. 22 shows another example of how the relay-facilitated communication configuration of FIG. 20 can be implemented.

FIG. 23 shows that in some embodiments, a relay can be implemented in a moving or movable device.

FIG. 24 shows that in some embodiments, the moving or movable device of FIG. 23 can be an aircraft such as an aerial drone configured to provide relay functionality.

FIG. 25 shows a system that includes multiple patches worn by respective cows, where a given patch is configured to communicate with an external device such as a relay device only if it has data to transmit.

FIG. 26 shows a system that includes multiple patches worn by respective cows, where a given patch is configured to communicate with an external device such as a relay device whenever interrogated by the external device.

FIGS. 27A to 27C show an example sequence of operation that can be implemented to perform the communication example of FIG. 25.

FIGS. 28A and 28B show examples of relay functionalities that can be implemented to relay the information obtained from one or more patches, such as in the example of FIGS. 27A to 27C.

FIG. 29 shows an example application where a mobile relay device such as an aerial drone can be particularly useful.

FIG. 30 shows a situation where position of a cow having a detected condition can be updated utilizing the mobile relay device.

FIG. 31 shows that in some embodiments, a mobile relay device such as an aerial drone can be configured to carry a payload to facilitate fertilization of a cow-in-heat.

FIG. 32 shows an example where a data set can be obtained for N cows through use of respective patches.

FIG. 33 show an example where locations and related data can be utilized to show clusters that may show how locations where cows are more likely to be in-heat.

FIG. 34 shows another example where the data set of FIG. 32 can be utilized to prioritize treatment of some cows over others.

FIG. 35 shows an example priority list that can be generated based on the example of FIG. 34.

FIG. 36 shows that in some embodiments, a communication component of a patch can be configured to provide a wireless communication with another device.

FIG. 37 shows that in some embodiments, a communication component of a patch can be configured to provide a wired communication with another device.

FIG. 38 shows a system that can be formed with one or more patches as described herein, and an external device.

FIG. 39 shows that in some embodiments, the system of FIG. 38 can include a plurality of patches that communicate with a common external device.

FIG. 40 shows that in some embodiments, the system of FIG. 38 can include a plurality of patches that can communicate with each other, and/or with an external device.

### DETAILED DESCRIPTION OF SOME EMBODIMENTS

The headings provided herein, if any, are for convenience only and do not necessarily affect the scope or meaning of the claimed invention.

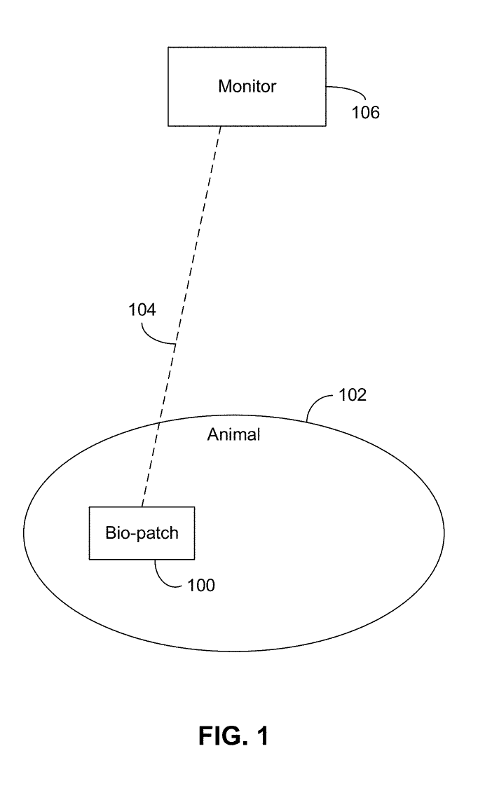

FIG. 1 depicts a bio-patch 100 (also referred to herein as simply a patch) having one or more features as described herein, being utilized for monitoring one or more biological conditions of, and/or one or more physical/behavioral activities associated with an animal 102. For the purpose of description, it will be understood that an animal can be a live animal such as a mammal. Such a mammal can be, for example, a livestock animal such as a cow. Although various examples are described in the context of cows, it will be understood that one or more features of the present disclosure can also be utilized for other types of livestock animals.

In the example of FIG. 1, the bio-patch 100 is shown to be capable of communicating (line 104) with a monitor 106. Various examples of such a communication are described herein in greater detail.

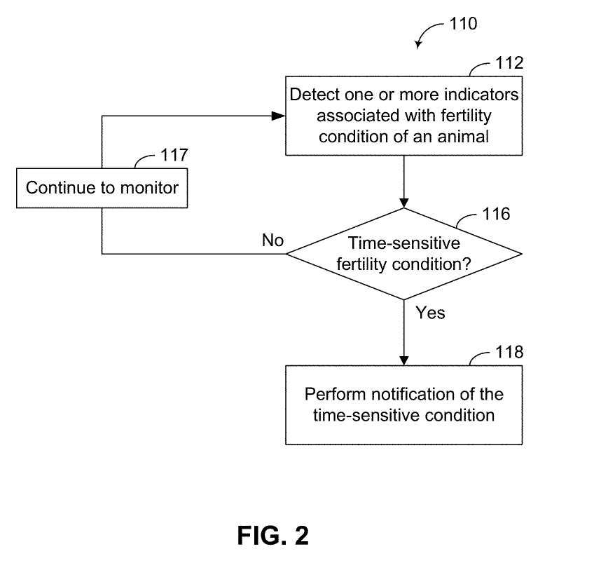

FIG. 2 shows a process 110 that can be implemented with a patch having one or more features as described herein. In block 112, one or more indicators associated with fertility condition of an animal can be detected with the patch. In a decision block 116, the process 110 can determine whether a time-sensitive fertility condition exists based on the detection of the one or more indicators. If No, the process 110 can continue to monitor the animal through block 117. If Yes, the process 110 can proceed to block 118 where notification of the time-sensitive condition can be performed. Examples related to some or all of the foregoing process and decision blocks are described herein in greater detail.

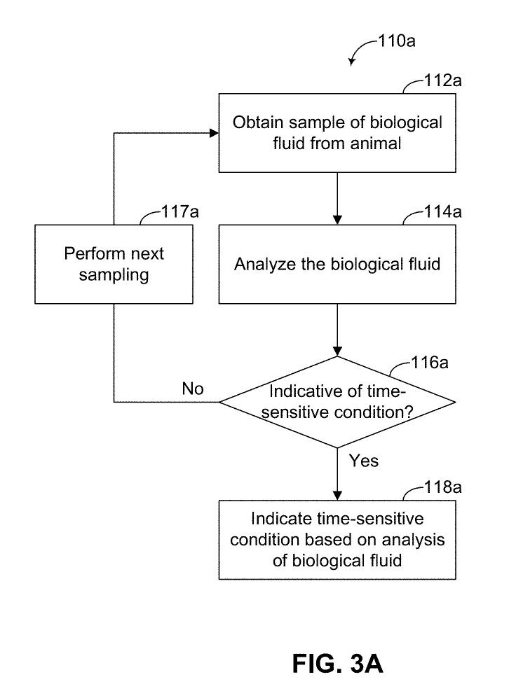

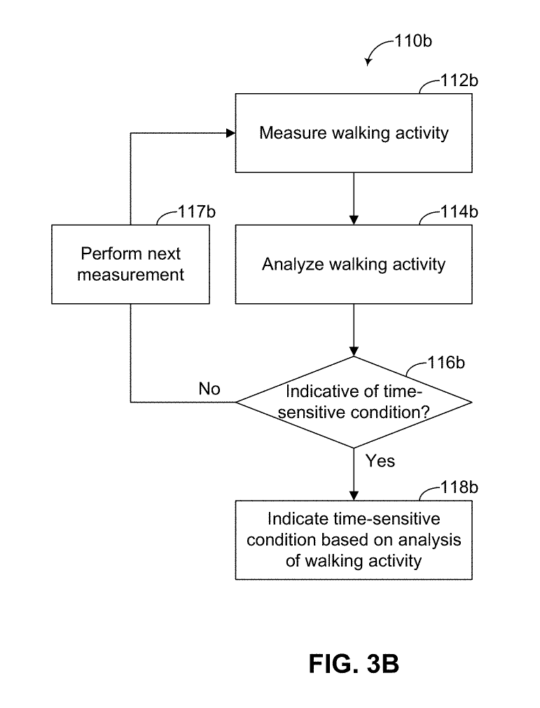

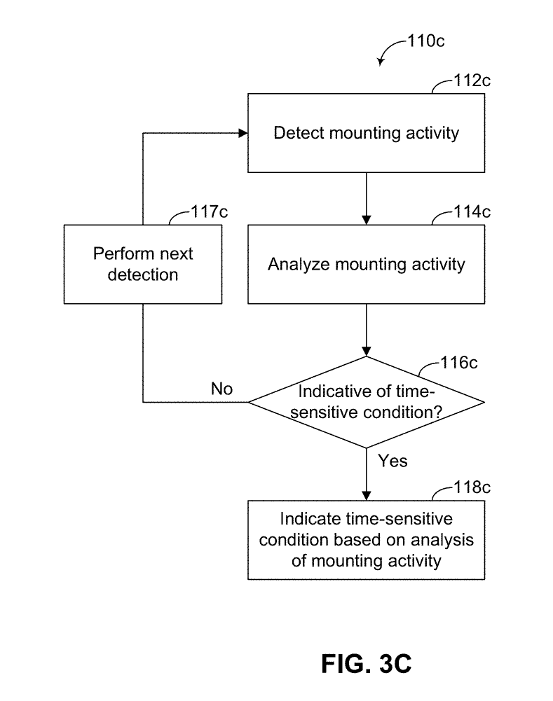

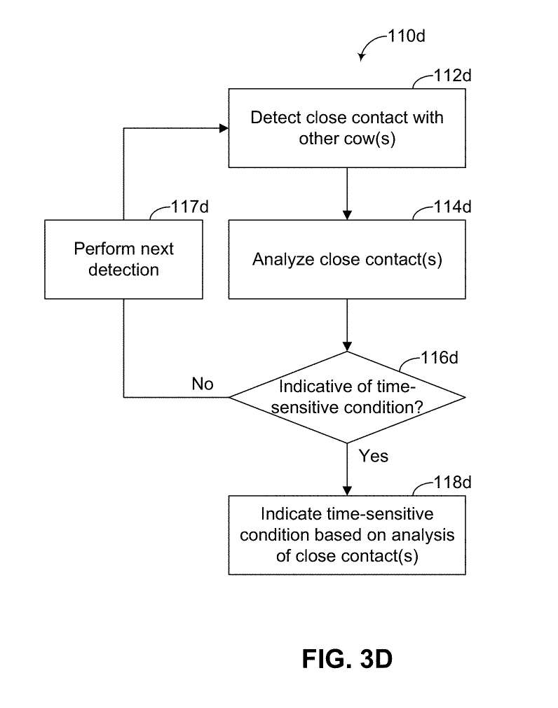

FIGS. 3A to 3D show more specific examples of the process 110 of FIG. 2. In some embodiments, such example processes of FIGS. 3A to 3D can be performed by a single patch, individual patches with each being configured specifically to provide respective functionality, or any combination thereof.

For example, FIG. 3A shows a process 110a that can be implemented with a patch having one or more features as described herein, based on analysis of a biological fluid. In block 112a, a sample of biological fluid (e.g., blood) can be obtained from an animal with the patch. In block 114a, at least some of the biological fluid can be analyzed. In a decision block 116a, the process 110a can determine whether a time-sensitive condition exists based on the analysis of the biological fluid. If No, the process 110a can repeat the biological fluid sampling and analysis steps continuously or after a selected time interval, through block 117a. If Yes, the process 110a can proceed to block 118a where notification of the time-sensitive condition can be performed. Examples related to some or all of the foregoing process blocks are described herein in greater detail.

In another example, FIG. 3B shows a process 110b that can be implemented with a patch having one or more features as described herein, based on analysis of physical activity. In block 112b, a measurement of physical activity such as walking activity level of an animal can be obtained with the patch. In block 114b, the measured walking activity level can be analyzed. In a decision block 116b, the process 110b can determine whether a time-sensitive condition exists based on the analysis of the walking activity level. If No, the process 110a can repeat the walking activity sampling and analysis steps continuously or after a selected time interval, through block 117b. If Yes, the process 110a can proceed to block 118b where notification of the time-sensitive condition can be performed. Examples related to some or all of the foregoing process blocks are described herein in greater detail.

In yet another example, FIG. 3C shows a process 110c that can be implemented with a patch having one or more features as described herein, based on analysis of physical activity. In block 112c, physical activity such as mounting activity of an animal can be detected with the patch. In block 114c, the detected mounting activity level can be analyzed. In a decision block 116c, the process 110c can determine whether a time-sensitive condition exists based on the analysis of the mounting activity detection. If No, the process 110c can repeat the mounting activity detection and analysis steps continuously or after a selected time interval, through block 117c. If Yes, the process 110c can proceed to block 118c where notification of the time-sensitive condition can be performed. Examples related to some or all of the foregoing process blocks are described herein in greater detail.

In yet another example, FIG. 3D shows a process 110d that can be implemented with a patch having one or more features as described herein, based on analysis of behavioral activity. In block 112d, behavioral activity such as a tendency of an animal to be in close contact with another animal can be detected with the patch. In block 114d, the detected close contact with one or more animals can be analyzed. In a decision block 116d, the process 110d can determine whether a time-sensitive condition exists based on the analysis of the detected close contact(s). If No, the process 110d can repeat the close-contact detection and analysis steps continuously or after a selected time interval, through block 117d. If Yes, the process 110d can proceed to block 118d where notification of the time-sensitive condition can be performed. Examples related to some or all of the foregoing process blocks are described herein in greater detail.

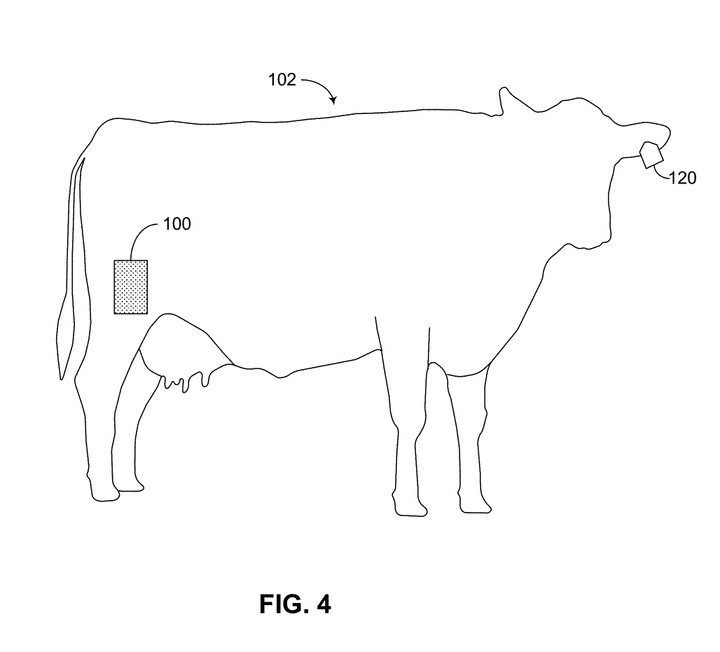

FIG. 4 depicts a cow 102 that can be a more specific example of the animal 102 of FIG. 1. In FIG. 4, the cow 102 is shown to have attached thereto a patch 100 having one or more features as described herein. It will be understood that the cow 102 can be provided with one or more patches each having one or more features as described herein. Such patch(es) be worn by the cow 102 at one or more different locations of the cow's body.

Suppose that the cow 102 is a dairy cow raised and maintained in a farm. Suppose further that such a dairy cow is utilized to reproduce, such that the number of cattle in the farm is maintained or increased over time. Accordingly, it is common for a given cow to be identifiable. For example, a tag 120 with a unique identifier (e.g., a number) can be secured to the cow 102. In some embodiments, the patch 100 worn by the cow 102 can be associated with the identifier (120), such that information provided by the patch 100 is associated with the cow 102.

It is noted that in many dairy farms, cows are allowed to roam and graze over a wide area. It is further noted that a time window suitable for reproduction is relatively small for a given cow. Thus, monitoring of reproductive cycles of many cows can be challenging, when conventional techniques (e.g., temperature measurements performed by a person) are utilized.

It is also noted that once a given cow is determined to be in a condition suitable for reproduction, artificial insemination is typically performed on the cow with stored bull semen. Such bull semen is typically expensive; thus, it is highly desirable to have such bull semen be utilized in an effective manner.

Based on the foregoing examples, and especially in the context of many cows that are allowed to roam and graze over a wide area, one can see that fertilizing cows in an effective manner can challenging when conventional techniques are utilized. In some embodiments, patches (such as the patch 100 of FIG. 4) having one or more features as described herein can detect and provide notification of cows' reproductive conditions with accuracy, and such information can be utilized to effectively fertilize reproduction-ready cows.

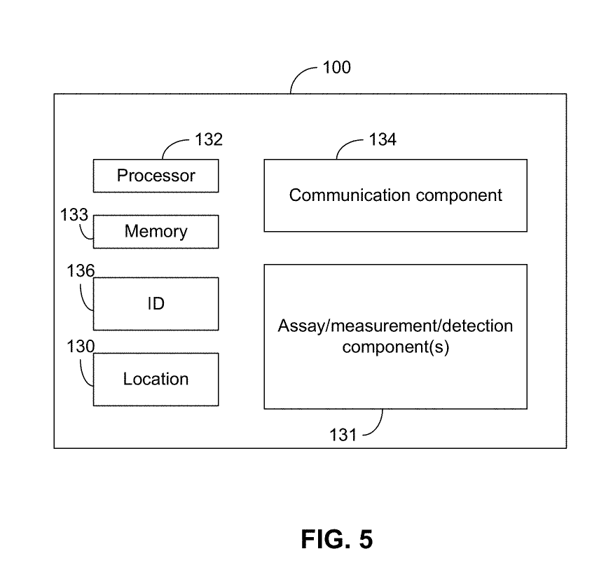

FIG. 5 shows that in some embodiments, a patch 100 having one or more features as described herein can include a number of components. For example, the patch 100 can include a component 131 configured to provide an assay, measurement and/or detection functionality, such as some or all of the examples of FIGS. 3A to 3D. The patch 100 can also include a communication component 134 configured to provide communication functionality to, for example, transfer information associated with fertility condition.

FIG. 5 also shows that in some embodiments, the patch 100 can further include a location-determining component 130 such as a GPS (global positioning system) circuitry. Such a circuitry can provide information about the location of the patch 100, and therefore, the location of the cow having the patch 100. In some embodiments, information transferred by the communication component 134 can include the location information.

FIG. 5 also shows that in some embodiments, the patch 100 can further include an identifier component 136 configured to provide information about the identity of the patch 100, and therefore, the identity of the cow having the patch 100. Such identity of the patch can be, for example, a unique number that can be correlated with the cow's tag (e.g., 120 in FIG. 4), programmable based on the cow's tag, etc. It will be understood that such an identity of the patch/cow can be implemented in a number of different ways. In some embodiments, information transmitted by the communication component 134 can include the foregoing identifier information.

FIG. 5 also shows that in some embodiments, some or all of the functionalities associated with the various components of the patch 100 can be controlled and/or facilitated by a processor 132. Such a processor can have some or all of the functionalities as described herein. In some embodiments, the patch 100 can also include a memory or storage component 133 (e.g., a non-transitory computer readable medium); and such a storage component can store, for example, algorithms and settings to support operation of the patch 100, data obtained by the operation of the patch 100, etc.

In some embodiments, the patch 100 of FIG. 5 can include a support structure configured to support some or all of the various components, such as the components shown in FIG. 5. Such a support structure can include one or more layers, and can be implemented in a number of wearable forms. Examples of such a support structure and different wearable forms are provided in U.S. Pat. No. 9,133,024 titled PERSONAL DIAGNOSTIC DEVICES INCLUDING RELATED METHODS AND SYSTEMS, which is expressly incorporated by reference in its entirely, and its disclosure is to be considered part of the specification of the present application.

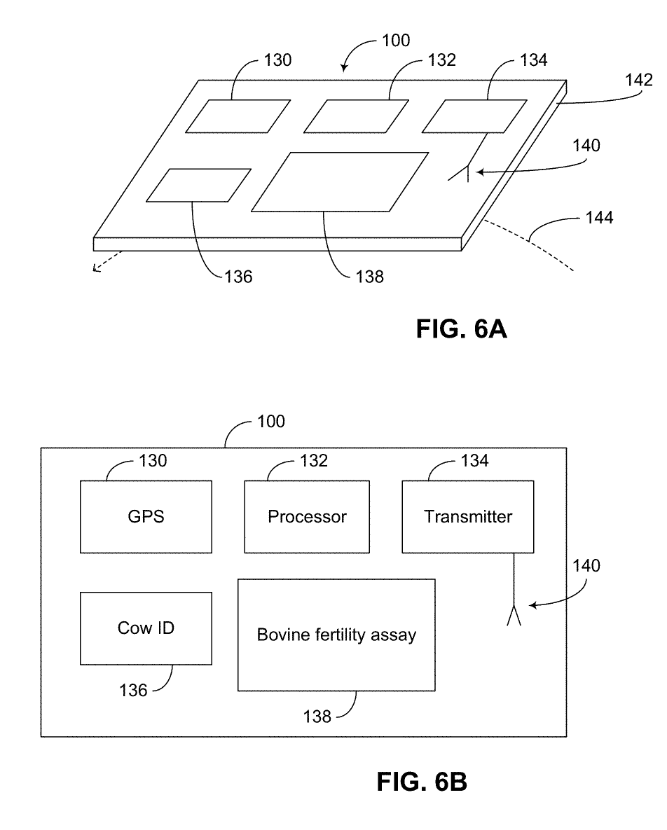

FIGS. 6A and 6B show a patch 100 that can be a more specific example of the patch 100 of FIGS. 2 and 5, and support the example process of FIG. 3A. More particularly, the patch 100 of FIGS. 6A and 6B can be configured to include a number of components implemented to allow, for example, detection of bovine fertility condition and notification of such a condition to a monitoring device or system. Accordingly, the patch 100 can include a component 138 configured to perform a bovine fertility assay as an example of the component 131 of FIG. 5, and a transmitter 134 configured to transmit information resulting from the bovine fertility assay. In embodiments where such transmission of information is achieved in a wireless manner, the patch 100 can further include an antenna 140 configured to support the transmission. Various examples related to the bovine fertility assay and the transmission of corresponding information are described herein in greater detail.

In some embodiments, the patch 100 of FIGS. 6A and 6B can further include a location-determining component 130 such as a GPS (global positioning system) circuitry. Such a circuitry can provide information about the location of the patch 100, and therefore, the location of the cow having the patch 100. In some embodiments, information transmitted by the transmitter 134 can include the location information.

In some embodiments, the patch 100 of FIGS. 6A and 6B can further include an identifier component 136 configured to provide information about the identity of the patch 100, and therefore, the identity of the cow having the patch 100. Such identity of the patch can be, for example, a unique number that can be correlated with the cow's tag (e.g., 120 in FIG. 4), programmable based on the cow's tag, etc. It will be understood that such an identity of the patch/cow can be implemented in a number of different ways. In some embodiments, information transmitted by the transmitter 134 can include the foregoing identifier information.

In some embodiments, some or all of the functionalities associated with the various components of the patch 100 of FIGS. 6A and 6B can be controlled and/or supported by a processor 132. Such a processor can have some or all of the functionalities as described herein. In some embodiments, the patch 100 of FIGS. 6A and 6B can also include a memory or storage component (e.g., a non-transitory computer readable medium); and such a storage component can store, for example, algorithms and settings to support operation of the patch 100, data obtained by the operation of the patch 100, etc.

In some embodiments, the patch 100 of FIGS. 6A and 6B can include a support structure 142 configured to support various components, such as the components shown in FIGS. 6A and 6B. Such a support structure can include one or more layers, and can be implemented in a number of wearable forms. Examples of such a support structure and different wearable forms can be found in the above-referenced U.S. Pat. No. 9,133,024.

In some embodiments, biological fluid such as blood can be obtained by the patch 100 of FIGS. 6A and 6B, and the bovine fertility assay can be performed with such a biological fluid. In some embodiments, such biological fluid can be sampled by the patch 100, where the sampling fluid terminates at the patch 100. In some embodiments, the patch can be configured to allow circulation of biological fluid therethrough, and sampling of the biological fluid for assay purpose can be obtained from such a circulating fluid. In the example of FIG. 6A, such a circulating fluid is depicted as 144.

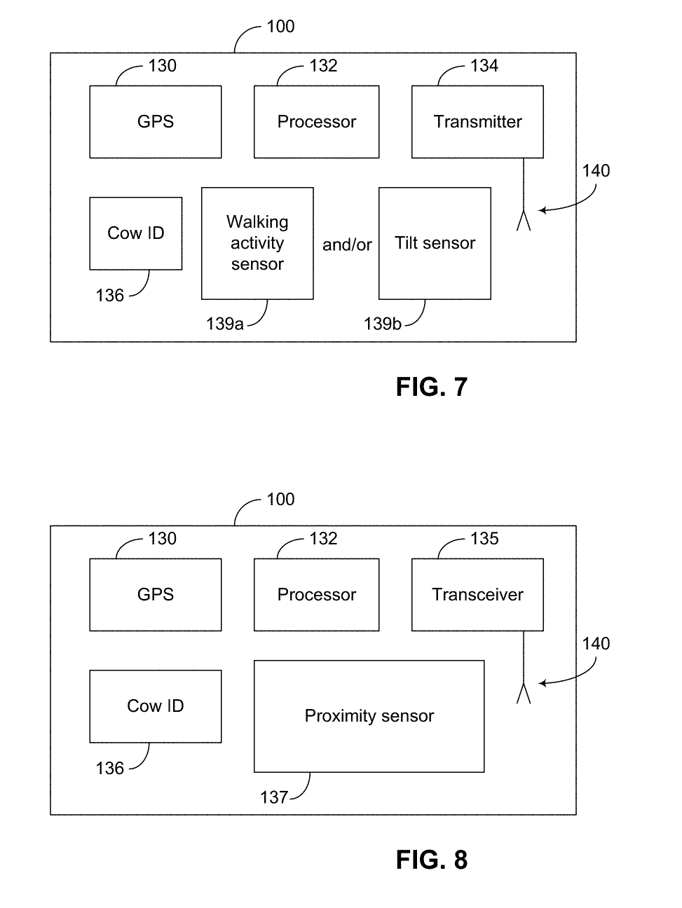

FIG. 7 shows a patch 100 that can be a more specific example of the patch 100 of FIGS. 2 and 5, and support either or both of the example processes of FIGS. 3B and 3C. More particularly, the patch 100 of FIG. 7 can be configured to include a number of components implemented to allow, for example, detection of physical activity(ies) and notification of such activity(ies) to a monitoring device or system. Accordingly, the patch 100 can include either or both of a walking activity sensor 139a and a tilt sensor 139b, and a transmitter 134 configured to transmit information associated with detected physical activity(ies).

For example, the walking activity sensor 139a can include a miniature pedometer having one or more accelerometers. Such a walking sensor can detect and count steps taken by a cow wearing the patch 100. It is noted that on the day of estrus, cows are typically much more active than usual, and such increased activity typically includes a significant increase in the number of steps (e.g., an increase up to a factor of four). Thus, detection of such an increase in the number of steps can provide an indication that the corresponding cow is in, or close to, an estrus time window.

In another example, the tilt sensor 139b can include a miniature sensor such as those utilized in small wireless devices. Such a tilt sensor can detect a tilt of a part of a cow wearing the patch 100. It is noted that when a cow is in estrus, it has a tendency to mount another cow, and/or be mounted by another cow or bull. If the cow wearing the patch 100 (e.g., worn on the side of the cow) mounts another cow, the tilt sensor 139b can detect the tilted orientation of the body of the mounting cow. If the cow wearing the patch 100 (e.g., worn on the tail) is mounted by another cow or bull, the tilt sensor 139b can detect the tilted orientation of the raised tail of the mounted cow. Thus, detection of such a tilt associated with the cow with the patch 100 can provide an indication that the cow is in, or close to, an estrus time window.

In embodiments where transmission of the foregoing information about the physical activity (such as step count and/or tilt) is achieved in a wireless manner, the patch 100 can further include an antenna 140 configured to support the transmission. In some embodiments, the patch 100 of FIG. 7 can further include a location-determining component 130 such as a GPS (global positioning system) circuitry. Such a circuitry can provide information about the location of the patch 100, and therefore, the location of the cow having the patch 100. In some embodiments, information transmitted by the transmitter 134 can include the location information.

In some embodiments, the patch 100 of FIG. 7 can further include an identifier component 136 configured to provide information about the identity of the patch 100, and therefore, the identity of the cow having the patch 100. Such identity of the patch can be, for example, a unique number that can be correlated with the cow's tag (e.g., 120 in FIG. 4), programmable based on the cow's tag, etc. It will be understood that such an identity of the patch/cow can be implemented in a number of different ways. In some embodiments, information transmitted by the transmitter 134 can include the foregoing identifier information.

In some embodiments, some or all of the functionalities associated with the various components of the patch 100 of FIG. 7 can be controlled and/or supported by a processor 132. Such a processor can have some or all of the functionalities as described herein. In some embodiments, the patch 100 of FIG. 7 can also include a memory or storage component (e.g., a non-transitory computer readable medium); and such a storage component can store, for example, algorithms and settings to support operation of the patch 100, data obtained by the operation of the patch 100, etc.

In some embodiments, the patch 100 of FIG. 7 can include a support structure configured to support various components, such as the components shown in FIG. 7. Such a support structure can include one or more layers, and can be implemented in a number of wearable forms. Examples of such a support structure and different wearable forms can be found in the above-referenced U.S. Pat. No. 9,133,024.

FIG. 8 shows a patch 100 that can be a more specific example of the patch 100 of FIGS. 2 and 5, and support the example process of FIG. 3D. More particularly, the patch 100 of FIG. 8 can be configured to include a number of components implemented to allow, for example, detection of a behavior-related activity and notification of such an activity to a monitoring device or system. Accordingly, the patch 100 can include a proximity sensor 137, and a transceiver 135 configured to support detection and notification of such behavior-related activity.

For example, the proximity sensor 137 can be configured to detect near presence of one or more cows wearing respective patches. Such near presence can be detected by, for example, location information provided by respective GPS information, a local connectivity feature (e.g., RFID circuitry or near-field circuitry), etc. It is noted that when a cow is in or close to estrus, it has a tendency to be near other cows. Thus, detection of such close proximity to one or more other cows can provide an indication that the corresponding cow is in, or close to, an estrus time window.

In embodiments where transmission of the foregoing information about the close proximity to other cow(s) is achieved in a wireless manner, the patch 100 can further include an antenna 140 configured to support the transmission. In some embodiments, the patch 100 of FIG. 8 can further include a location-determining component 130 such as a GPS (global positioning system) circuitry. Such a circuitry can provide information about the location of the patch 100, and therefore, the location of the cow having the patch 100. In some embodiments, information transmitted by the transceiver 135 can include the location information.

In some embodiments, the patch 100 of FIG. 8 can further include an identifier component 136 configured to provide information about the identity of the patch 100, and therefore, the identity of the cow having the patch 100. Such identity of the patch can be, for example, a unique number that can be correlated with the cow's tag (e.g., 120 in FIG. 4), programmable based on the cow's tag, etc. It will be understood that such an identity of the patch/cow can be implemented in a number of different ways. In some embodiments, information transmitted by the transmitter 134 can include the foregoing identifier information.

In some embodiments, some or all of the functionalities associated with the various components of the patch 100 of FIG. 8 can be controlled and/or supported by a processor 132. Such a processor can have some or all of the functionalities as described herein. In some embodiments, the patch 100 of FIG. 8 can also include a memory or storage component (e.g., a non-transitory computer readable medium); and such a storage component can store, for example, algorithms and settings to support operation of the patch 100, data obtained by the operation of the patch 100, etc.

In some embodiments, the patch 100 of FIG. 8 can include a support structure configured to support various components, such as the components shown in FIG. 8. Such a support structure can include one or more layers, and can be implemented in a number of wearable forms. Examples of such a support structure and different wearable forms can be found in the above-referenced U.S. Pat. No. 9,133,024.

It will be understood that the examples described herein in reference to FIGS. 6 to 8 are functionalities that can be implemented in a single patch, in different individual patches, or some combination thereof. For example, the patch 100 of FIG. 5 can include all of the functionalities associated with FIGS. 6 to 8. In such an example, the component 131 of FIG. 5 can include the component 138 of FIGS. 6A and 6B, the components 139a and 139b of FIG. 7, and the component 137 of FIG. 8.

In another example the patch 100 of FIG. 5 can include some of the functionalities associated with FIGS. 6 to 8. For example, a combination of functionalities can include the component 138 of FIGS. 6A and 6B and either or both of the components 139a and 139B of FIG. 7. In another example, a combination of functionalities can include the component 138 of FIGS. 6A and 6B and the component 137 of FIG. 8. In yet another example, a combination of functionalities can include either or both of the components 139a and 139b of FIG. 7 and the component 137 of FIG. 8.

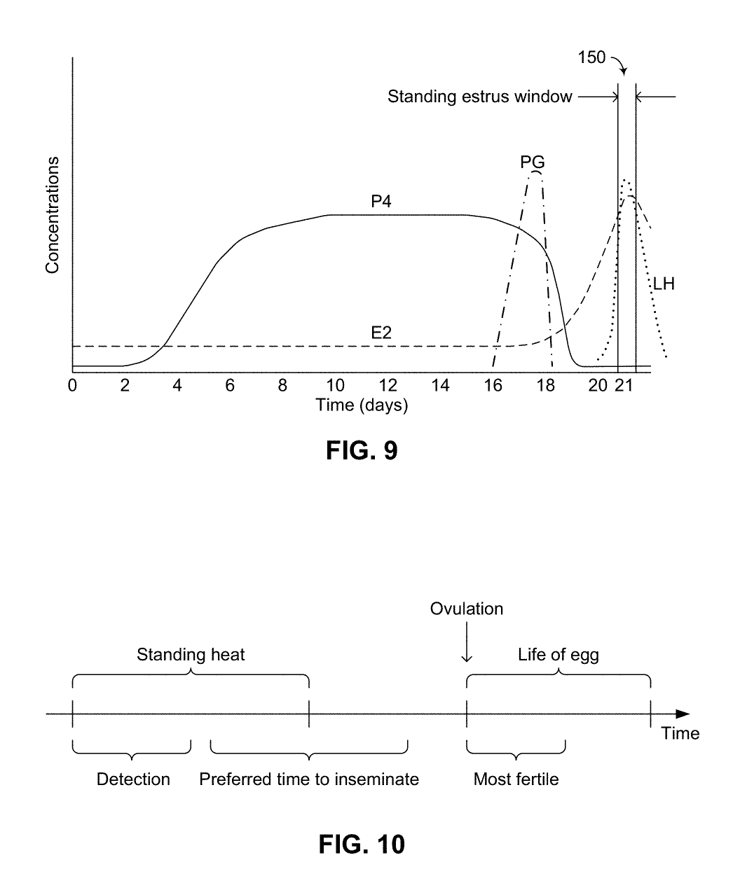

FIGS. 9-15 show examples of how bovine fertility assay can be performed by a patch having one or more features as described herein. FIG. 9 shows plots of time-dependent concentration levels of example hormones present in cows. For the purpose of description, suppose that a given cow's estrous cycle is approximately 21 days, with a standing estrus window 150 occurring on or around day 21. It is noted that such a standing estrus window typically lasts about 15 hours, but can range from a much shorter duration less than 6 hours to a longer duration of about 24 hours. During such a standing estrus window, the female cow is typically ready to be bred, either by being mounted by a bull or by being artificially inseminated.

Referring to the example of FIG. 9, it is noted that progesterone (P4, inhibits ovulation) level in the example cow begins to rise at around day 4, reaches and remains at a relatively high level until around day 17, and decreases to a relatively low level by day 19. The P4 level remains at the relatively low level during the standing estrus window 150.

Referring to the example of FIG. 9, it is noted that estradiol (E2) level in the example cow remains relatively low between day 0 and day 17, begins to rise at around day 18, peaks at a time within the standing estrus window 150, and begins to decrease at the end of the standing estrus window 150. It is noted that an elevated concentration of E2 in the absence of P4 typically causes behavioral changes associated with standing estrus and causes release of a surge of gonadotropin releasing hormone (GnRH). It is further noted that GnRH typically causes a preovulatory surge of luteinizing hormone (LH) to occur, resulting in ovulation of an ovulatory follicle.

Referring to the example of FIG. 9, it is noted that prostaglandin F_{2α} (PG) level in the example cow remains relatively low during most days of the cycle. On around day 16, the PG level begins to increase, and the PG level peaks on around day 18, and decreases rapidly thereafter.

Referring to the example of FIG. 9, it is noted that luteinizing hormone (LH) level in the example cow also remains relatively low during most days of the cycle. On around day 20, the LH level begins to increase relatively fast, and the LH level peaks within the standing estrus window 150, and then decreases relatively fast thereafter.

Based on the foregoing examples, FIG. 10 shows an example timeline near the standing estrus window 150 of FIG. 9. In FIG. 10, the standing estrus window 150 of FIG. 9 is indicated as a standing heat period. Such a period can last about 15 to 18 hours. Ovulation occurs after such a standing heat period, and the resulting fertile egg has a life of about 10 to 20 hours, with the most fertile portion being about 8 to 10 hours following ovulation. Considering that bull's sperm has a life in the cow's reproductive tract of about 18 to 24 hours, a preferred time to inseminate the cow begins roughly about halfway in the standing heat period and ends at a time roughly between the end of the standing heat period and the ovulation time. Accordingly, detection of the standing heat condition before the foregoing preferred insemination period is highly desirable. It is noted that such a detection period is relative short. It is further noted that a patch having one or more features as described herein can allow such detection in an efficient manner.

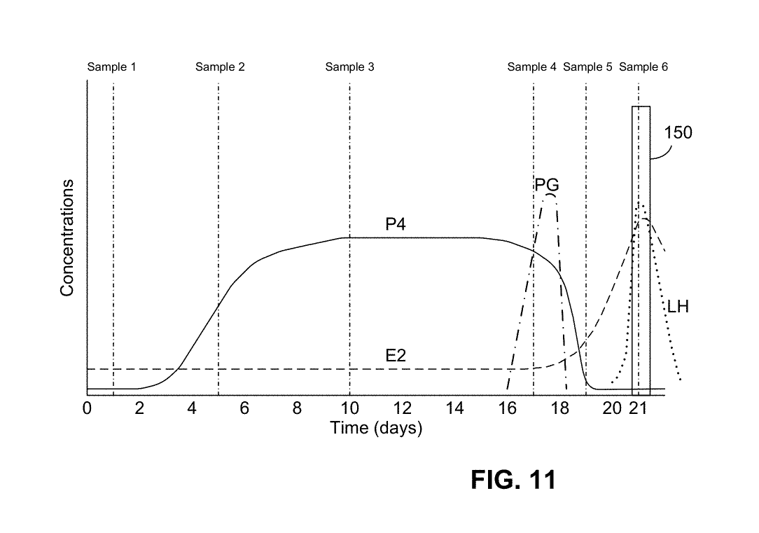

FIG. 11 shows examples of samplings that can be performed at different times in the example fertility cycle of FIG. 9. It will be understood that with each sampling, concentration(s) of some or all of the example hormones can be determined or estimated. It will also be understood that such sampled hormones can include those depicted in FIG. 9, and/or other reproduction related hormones present in cows.

In the example of FIG. 11, first sampling (Sample 1) is shown to be performed on day 1; second sampling (Sample 2) is shown to be performed on day 5; third sampling (Sample 3) is shown to be performed on day 10; fourth sampling (Sample 4) is shown to be performed on day 17; fifth sampling (Sample 5) is shown to be performed on day 19; and sixth sampling (Sample 6) is shown to be performed on day 21. With such example samplings, some or all of representative hormone concentrations listed in Table 1 can be obtained.

TABLE 1

| | | E2 | | | |
|---|---|---|---|---|---|
| Sample | P4 level | level | PG level | LH level | Likely day(s) in cycle |
| 1 | Low | Low | Low | Low | 1-3 |
| 2 | Medium | Low | Low | Low | 4-6 |
| 3 | High | Low | Low | Low | 7-16 |
| 4 | High | Low | High | Low | 17, 18 |
| 5 | Low | Low | Low | Low | 19 |
| 6 | Low | High | Low | High | 21 |

In the examples of FIG. 11 and Table 1, the concentration levels of each hormone are characterized as being low, medium or high. It will be understood that such concentration levels can also be characterized with greater or lesser granularity. It will also be understood that some or all of the hormones can be characterized quantitatively, qualitatively, or any combination thereof.

Referring to the examples of FIG. 11 and Table 1, it is noted that there can be ambiguities in likely cycle days when one considers combinations of hormone levels. For example, Sample 1 corresponding to likely days 1-3 has all four hormones being low, similar to Sample 5 corresponding to likely day 19. However, such ambiguity can be removed by one or more samplings before and/or after a given sampling.

It is also noted that when a new sampling is performed, and especially if such a sampling happens to result in an ambiguity, one may not know what part of the cycle corresponds to the sampling. In some embodiments, one or more further samplings can be utilized to better determine where the cow is in the current cycle.

In some embodiments, the new sampling can be combined with additional information to allow determination of where the cow is in the current cycle, without any additional sampling(s). For example, a patch having one or more features as described herein can be configured to measure one or more additional properties of the cow (e.g., body temperature, heart rate, physical/behavioral activity(ies), etc.) other than hormone levels. Such additional property(ies) can be combined with the hormone measurement(s) to allow better determination of where the cow is in the current cycle.

In another example, a person applying a patch to a cow may have information about the last standing estrus window. In some embodiments, the patch can be provided with such information; and the results from the new sampling can be combined with such input-information to allow better determination of where the cow is in the current cycle.

In some embodiments, a patch having one or more features as described herein can be configured to sample and analyze a cow's bodily fluid such as blood in a number of different ways. For example, such sampling and analysis of bodily fluid can be performed on a regular interval (e.g., more than once per day, daily, once every N days (N>1), etc.). It is noted that if the sampling/analysis frequency is higher, time-progression of the cow's fertility cycle can be monitored with greater precision; however, such higher sampling/analysis frequency can be challenging if there is a limited supply of resources such as analysis chemicals, power, etc. On the other hand, if the sampling/analysis frequency is lower, time-progression of the cow's fertility cycle can be monitored with lesser precision; however, such lower sampling/analysis frequency can allow the patch to operate longer if there is a limited supply of resources such as analysis chemicals, power, etc.

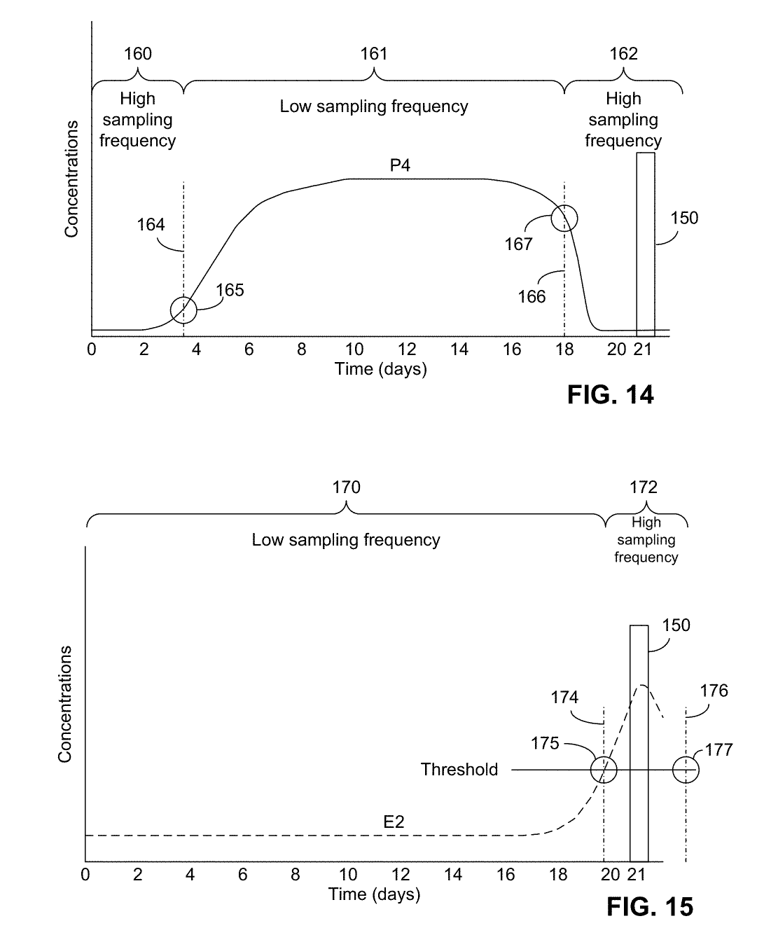

In some embodiments, a patch having one or more features as described herein can be configured to have variable sampling frequencies. Such variable sampling frequencies can allow more meaningful samplings to be performed during one or more time periods, and less samplings to be performed during time period(s) when, for example, hormone levels are not likely to change significantly. Examples related to such variable sampling frequencies are described in greater detail in reference to FIGS. 14 and 15.

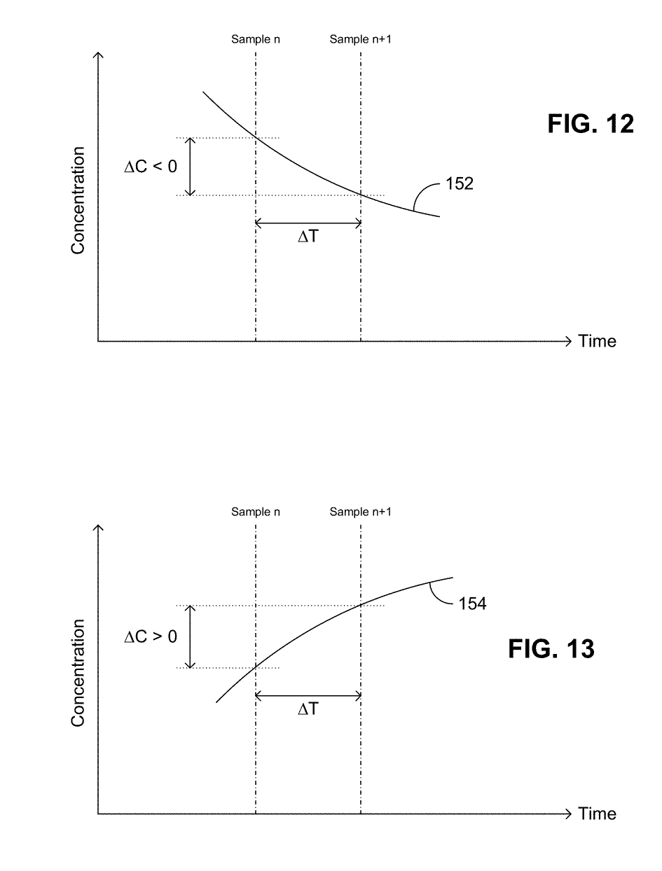

In the examples of FIG. 11 and Table 1, a given fertility cycle is characterized with various concentration levels of hormones. FIGS. 12 and 13 show that in some embodiments, a patch having one or more features as described herein can be configured to make measurements of, for example, changes in hormone levels. Aside from obtaining information about changes in hormone levels, such measurements can allow systematic errors to be canceled out or reduced.

For example, each of FIGS. 12 and 13 shows a measurement situation where two measurements are made as Sample n and Sample n+1, separated by a time interval ΔT. With the two measurements, a change in concentration level (ΔC) of a hormone over the time interval ΔT can be determined. Such a change (ΔC/ΔT) can provide an estimate of a time-dependent trend of an actual concentration level of the hormone. In the example of FIG. 12, such a time-dependent actual concentration level is depicted as 152, and is shown to decrease during the time period ΔT. In the example of FIG. 13, such a time-dependent actual concentration level is depicted as 154, and is shown to increase during the time period ΔT.

In some embodiments, the foregoing measurement of time-dependent change in concentration can be utilized to predict when the standing estrus window will likely occur. Referring to FIGS. 9 and 11, it is noted that significant changes in hormone concentrations occur several days prior to the standing estrus window 150. For example, the level of P4 decreases significantly between days 17 and 19. In another example, the level of PG increases significantly between days 16 and 17.5, and then decreases sharply until day 18. In yet another example, the level of E2 begins to increase significantly from day 18, and peaks at around day 21. Thus, one or more of the foregoing changes in hormone concentrations can be detected to predict the occurrence of the standing estrus window 150.

In some embodiments, a patch having one or more features as described herein can be configured to provide variable sampling frequencies. For example, FIG. 14 shows a sampling configuration where P4 can be sampled with different frequencies, depending on detection of an increase and a decrease in the P4 level. More particularly, when an increase in P4 level that exceeds a threshold increase-rate is detected, such a detection can be determined to be at a beginning portion of a relatively lengthy period of high P4 level. In FIG. 14, such a detection is indicated as 165 at a time 164 between days 3 and 4. Preceding such a detection is a relatively short period of about 5 days (e.g., day 19 to day 3) that includes the standing estrus window. Accordingly, during such a short period, sampling of P4 and/or one or more other hormone levels can be achieved with a higher frequency.

Following the detection 165 at time 164, a relatively long period of about 14 days (e.g., day 4 to day 18) starts, during which P4 level increases to a high level and then decreases. Similar to the detection 165 in the increase of P4 level, when a decrease in P4 level that exceeds a threshold decrease-rate is detected, such a detection can be determined to be at or near the ending portion of the relatively lengthy period of high P4 level. In FIG. 14, such a detection is indicated as 167 at a time 166 at about day 18. Accordingly, during such a long period 161 that does not include the standing estrus window 150, sampling of P4 and/or one or more other hormone levels can be achieved with a lower frequency.

Referring to FIG. 14, higher frequency sampling can be implemented during a period 162 that includes the standing estrus window 150. Such a period (162) can begin at or near the detection 167 at time 166, and end at the detection 165 at time 164. In the example of FIG. 14, the higher frequency sampling period 162 and a period indicated as 160 are parts of the same sampling period, due to the cyclical nature of the fertility cycle.

In the example of FIG. 14, the beginning and end of the lower-frequency sampling period 162 are based on detection of increasing and decreasing rates of a given hormone (e.g., P4 hormone). It will be understood that absolute values of such increasing and decreasing rates may or may not be the same.

The example of FIG. 14 relates to a situation where sample frequency can be adjusted based on detection of rates of changes in a selected hormone level. In some embodiments, a patch having one or more features as described herein can be configured to provide different sampling frequencies based on one or more concentration levels of a selected hormone. In some embodiments, a patch having one or more features as described herein can be configured to provide different sampling frequencies based on one or more indicators other than those obtained from analysis of hormones.

FIG. 15 shows a plot of the E2 hormone, where the concentration level peaks at or near the estrus window 150. The E2 hormone level is also shown to be at a low level for most of the fertility cycle. Thus, during such a low-level period 170, sampling can be performed with a relatively low frequency. Once the detected level of E2 hormone is higher than a first threshold value (e.g., detection 175 at time 174), sampling frequency can be increased; and such a higher-frequency sampling period (172) can be maintained until the detected level of E2 hormone is lower than a second threshold value (e.g. detection 177 at time 176).

In the example of FIG. 15, the higher-frequency sampling period 172 is shown to include the standing estrus window 150. Accordingly, during such a period (and especially prior to the standing estrus window 150), more precise estimate of the timing of the standing estrus window 150 can be obtained.

In the example of FIG. 15, the beginning and end of the higher-frequency sampling period 172 are depicted as being based on detection of a common threshold concentration level value for a given hormone (e.g., E2 hormone). However, it will be understood that the first threshold value (associated with the beginning of the period 172) may or may not be the same as the second threshold value (associated with the end of the period 172).

In FIG. 14, an example is provided where a sampling frequency adjustment can be based on detection of rates of changes in concentration levels of a given hormone. In FIG. 15, an example is provided where a sampling frequency adjustment can be based on detection of concentration levels of a given hormone. It will be understood that in some embodiments, a sampling frequency adjustment can be based on some combination of rate(s) of change(s) in concentration level(s) and actual concentration level(s) of one or more hormones.

In the examples of FIGS. 14 and 15, various references are made to sampling frequencies and detections of events that trigger changes in sample frequencies. It will be understood that when in a given sampling frequency period, the detection event that triggers transition to another sampling frequency may or may not coincide with a transition condition. For example, referring to FIG. 15, when the detection 175 occurs at time 174, the actual measured E2 hormone level may have already exceeded the selected threshold level. Thus, the actual detection time may be some time after the ideal time corresponding to the selected threshold level.

Accordingly, in some embodiments, a sampling frequency in a given period can be selected to provide sufficient time between the detection time and the beginning of the standing estrus window 150. For example, during a lower-frequency sampling period 170 of FIG. 15, sampling can be performed once or twice a day (e.g., based on an internal clock of the patch, at appropriate time(s) to obtain accurate measurement(s)). In the example of FIG. 15, if such a sampling is performed once a day, detection can be achieved on day 20 to provide at least one day between the detection and the beginning of the standing estrus window 150. During the higher-frequency sampling period, sampling can be performed, for example, at double the frequency of the lower-frequency sampling period. Such an increased sampling frequency can provide a more accurate estimate of the timing of the standing estrus window 150.

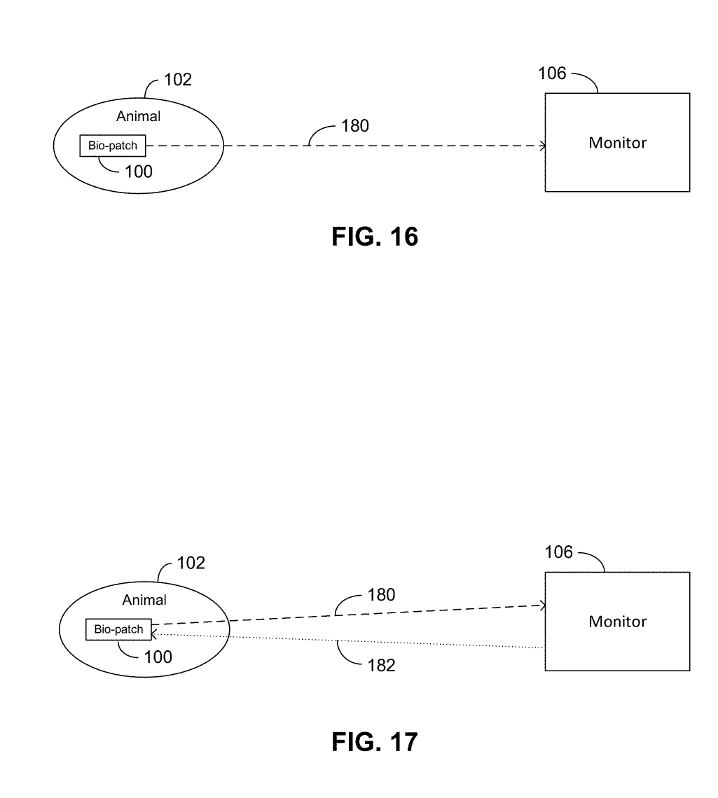

As described herein in reference to FIGS. 5 to 8, a patch having one or more features as described herein can include a transmitter to support transmission of information such as some or all of the sampling and/or detected data described herein. FIG. 16 shows an example of a system that can be implemented to utilize such a transmission functionality. For example, a patch 100 having one or more features as described herein is shown to be worn by an animal 102 such as a cow. Information transmitted (e.g., in a wireless manner) is depicted as 180, and such information can be received by a monitor 106. Such a monitor can include a receiver circuit configured to process the received signal from the patch 100. The monitor 106 can further include a processor to facilitate, for example, notification of an approaching standing estrus window.

In some embodiments, a patch having one or more features as described herein can also include a receiver circuit to allow the patch to receive information such as instructions, diagnostics, etc. Accordingly, FIG. 17 shows an example of a system that can be implemented to utilize such transmit and receive functionalities. For example, a patch 100 having one or more features as described herein is shown to be worn by an animal 102 such as a cow. Information transmitted (e.g., in a wireless manner) is depicted as 180, and such information can be received by a monitor 106. Such a monitor can include a receiver circuit configured to process the received signal from the patch 100. The monitor 106 can further include a processor to facilitate, for example, notification of an approaching standing estrus window.

In the example of FIG. 17, the patch 100 can also receive information (indicated as 182). Such received information can be achieved in a wireless mode, a wire mode, or any combination thereof. Although such information is depicted as being provided by the monitor 106, it will be understood that information provided to the patch 100 may or may not be from the same component (e.g., monitor 106 in FIG. 17).

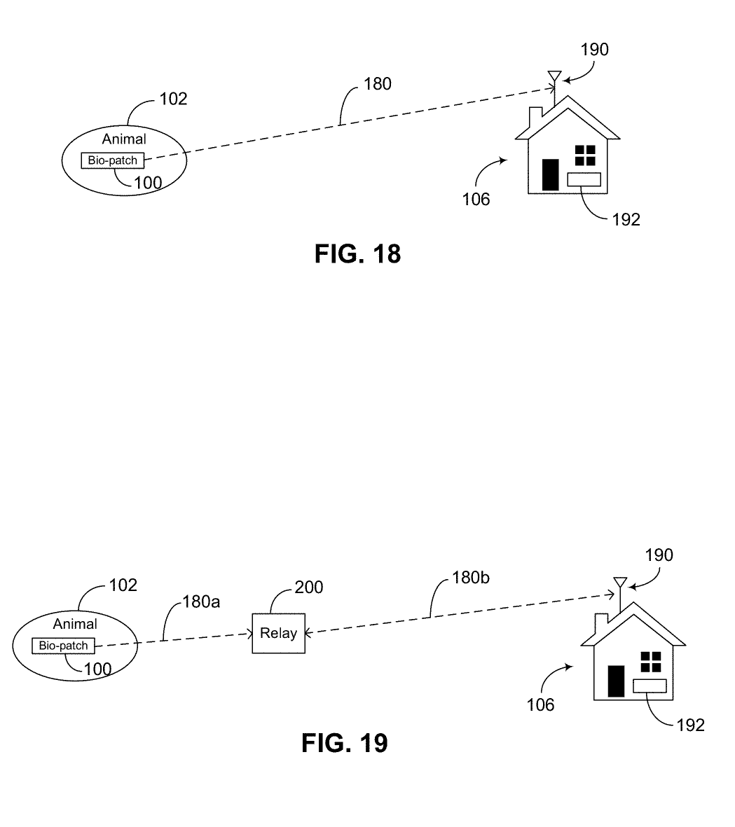

In the examples of FIGS. 16 and 17, the monitor 106 can be a single device, an assembly of a plurality of devices, a system, etc. configured to provide respective functionalities. For example, FIGS. 18 and 19 show that a monitor 106 can include an electronic device 192 having at least a receiver functionality and a processor. Such an electronic device can be in a building, and be in communication with an antenna 190. Accordingly, a signal 180 from the patch 100 (worn by the animal 102) can be received by the antenna 190 and processed by the electronic device 192.

In the example of FIG. 18, the patch 100 can communicate directly with the electronic device 192 of the monitor 106. In the example of FIG. 19, the patch 100 can communicate with the electronic device 192 through one or more relays 200. In such a configuration, the relay(s) 200 may or may not be considered to be part of the monitor 106.

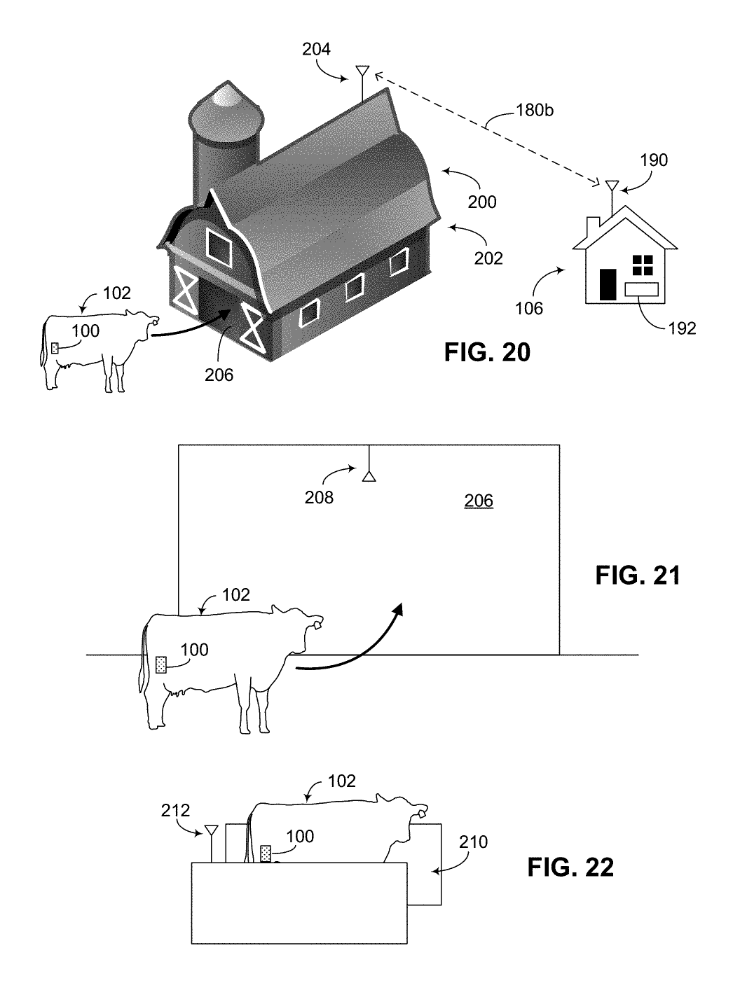

FIG. 20 shows a more specific example of the relay-facilitated communication configuration of FIG. 19. In the example of FIG. 20, a cow 102 wearing a patch 100 is shown to be entering a building 202 through an entrance 206. Such a building can be utilized to, for example, provide shelter, milking process, etc. In some embodiments, the building 202 can include one or more communication components so as to provide a relay functionality between the patch 100 and a monitor 106 (e.g., at a separate building).

More particularly, and referring to FIGS. 20 and 21, an antenna 208 can be provided at or near the entrance 206 of the building 202, so that when the cow 102 passes through the entrance 206, communication occurs between the patch 100 and the antenna 208. Such communication can be, for example, radio-frequency (RF) communication based on an RFID circuit in the patch 100. In such a configuration, the proximity of the patch 100 to the antenna 208 can result in the RFID circuit being activated and read by a wireless interaction between the antenna 208 and the RFID circuit. Information transferred from the patch 100 to the antenna 208 can include, for example, fertility assay data, and/or detected data associated physical/behavioral activity(ies), including one or more of the examples described herein.

Referring to FIGS. 20 and 21, the antenna 208 can communicate directly with the electronic device 192 of the monitor 106, through another antenna 204, through a wire, or any combination thereof. In FIG. 20, an example communication between the antenna 208 (via another antenna) and the electronic device 192 (via an antenna 190) is depicted as 180b.

The foregoing example of information being read out of the patch 100 at or near the entrance 206 is one of a number of situations during which communication can occur between the patch and the monitor 106. For example, FIG. 22 shows that in some embodiments, an antenna 212 can be provided at or near a stall 210 where a cow 102 with a patch 100 remains generally stationary for some time. For example, such a stall can be utilized to allow the cow 102 to rest, milk the cow 102, etc. The antenna 212 can provide communication functionalities similar to the example of FIG. 21.

In the examples described above in reference to FIGS. 20-22, the cow 102 is either entering a building or within the building. It will be understood that similar communication functionality can be implemented as a cow is exiting a building.

In the examples described above in reference to FIGS. 20-22, the example antennas (204 in FIG. 20, 208 in FIG. 21, 212 in FIG. 22) and related circuits/devices can be considered to be parts of a relay component (200 in FIG. 19). Such a relay in the example of FIGS. 20-22 is generally stationary since it is associated with a stationary building.

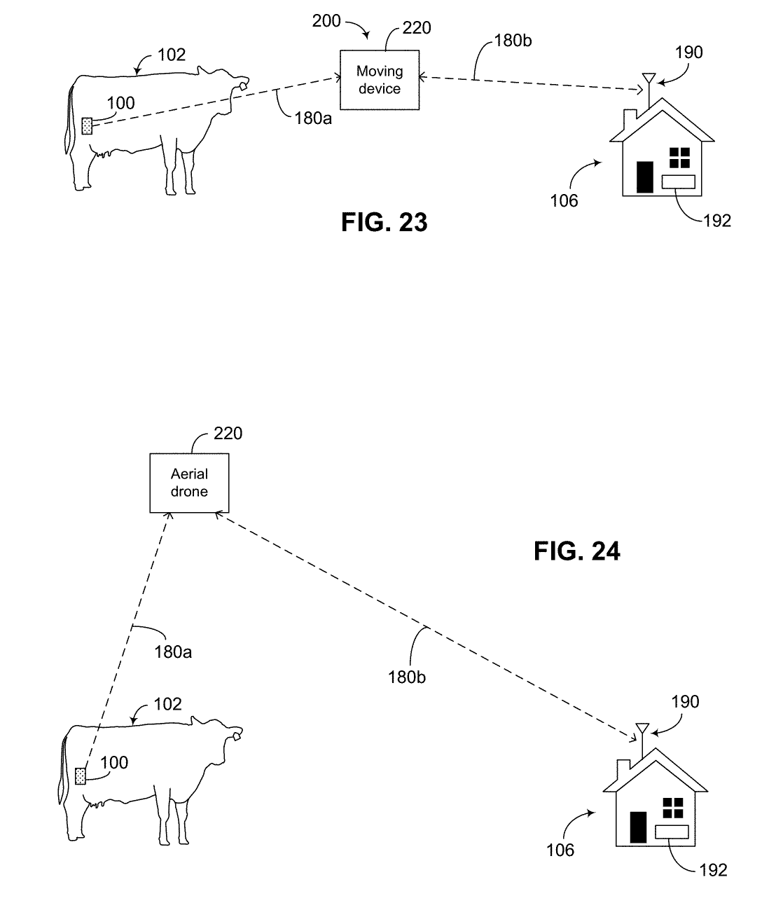

FIG. 23 shows that in some embodiments, a relay 200 can be implemented in a moving or movable device 220. Such a relay can provide communication functionalities similar to the example of FIG. 19, between the patch 100 and the monitor 106. In some embodiments, the moving or movable device 220 can be, for example, a ground vehicle, a watercraft, an aircraft, or a satellite. For each of earth-bound examples, including the aircraft, such a moving or movable platform can be configured to be controlled by a human operator, controlled without a human operator, or any combination thereof.

For example, FIG. 24 shows an example of an aircraft such as an aerial drone 220 configured to provide the relay functionality of FIG. 23. In FIG. 24, the aerial drone 220 is depicted as flying or generally hovering over a cow 102 with a patch 100, so as to communicate (180a) with the patch 100. Some or all of such communication can be relayed (180b) to and/or from a monitor 106.

In the example of FIG. 24, the communication 180a between the patch 100 and the aerial drone 220 is depicted as a one-way communication (e.g., data being read out of the patch 100). However, it will be understood that such communication between the patch 100 and the aerial drone 220 can be implemented as a two-way communication.

In the example of FIG. 24, the communication 180b between the aerial drone 220 and the monitor 106 is depicted as being a two-way communication. A communication from the aerial drone 220 to the monitor 106 can include, for example, data read out from the patch 100, information related to the operation of the aerial drone 220, etc. A communication from the monitor 106 to the aerial drone 220 can include, for example, flight control signals, etc.

It will be understood that the communication between the aerial drone 220 and the monitor 106 does not necessarily need to be a two-way communication. For example, an aerial drone can be programmed to perform a selected flight, and during such a flight relay of information from one or more patches can be achieved without additional control input to the aerial drone.

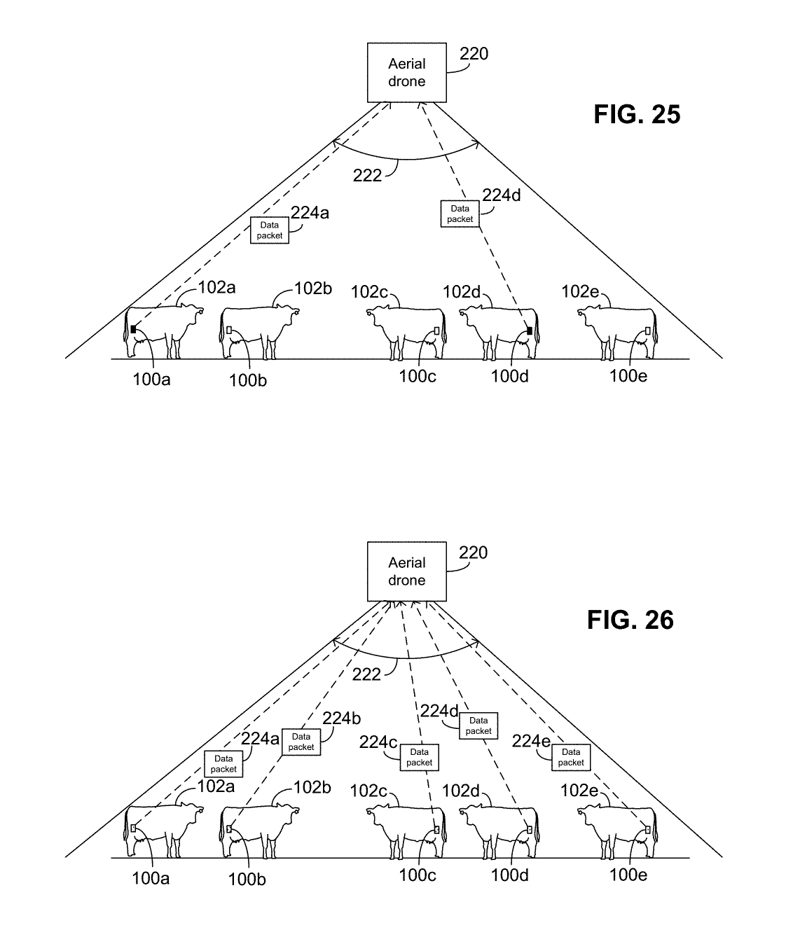

In some applications, the aerial mobility capability of an aerial drone can be particularly useful for monitoring cows that may be a remote locations and/or spread out in a large area. Examples related to use of such an aerial drone are described in reference to FIGS. 25-31.

Among others, FIGS. 25 and 26 show that in some embodiments, a patch having one or more features as described herein can be configured to communicate with an external device (e.g., a relay device such as an aerial drone) only under a selected condition (e.g., if there is an assay result to transmit), whenever interrogated by an external device, or any combination thereof. For example, FIG. 25 shows a system that includes multiple patches (100a-100e) worn by respective cows (102a-102e), where a given patch is configured to communicate with an external device such as a relay device (e.g., an aerial drone 220) only if it has assay data to transmit. Suppose that in the example of FIG. 25, the patches 100a, 100d worn by the respective cows 102a, 102d have assay data to transmit, regarding current fertility condition of the cows (102a, 102d), and the other patches have no such data to transmit. Accordingly, each of the two example patches 100a, 100d can transmit a respective data packet (224a or 224d) to the aerial drone 220 positioned over the respective cows 102a, 102d. However, the other patches (100b, 100c, 100e) do not transmit data, since they do not have any new fertility conditions of their respective cows. In some embodiments, such transmission can occur when the aerial drone 220 sends out one or more interrogation signals in a range 222 covered by the aerial drone 220.

In another example, FIG. 26 shows a system that includes multiple patches (100a-100e) worn by respective cows (102a-102e), where a given patch is configured to communicate with an external device such as a relay device (e.g., an aerial drone 220) whenever interrogated by the external device. Suppose that in the example of FIG. 26, each of the cows 102a, 102d has a current fertility condition detected by the respective patch, and the other do not have such current fertility conditions. Accordingly, each of the two example patches 100a, 100d can transmit a respective data packet (224a or 224d) to the aerial drone 220 positioned over the respective cows 102a, 102d, and such data packet can include data related to the current fertility condition detected by the respective patch. The other patches (100b, 100c, 100e) can also transmit respective data packets (224b, 224c, 224e) to the aerial drone 220, but such data packets do not have data related to any new fertility conditions of their respective cows. In some embodiments, the data packets 224b, 224c, 224e can include, for example, status of the patches. In some embodiments, the transmissions in the example of FIG. 26 can occur when the aerial drone 220 sends out one or more interrogation signals in a range 222 covered by the aerial drone 220.

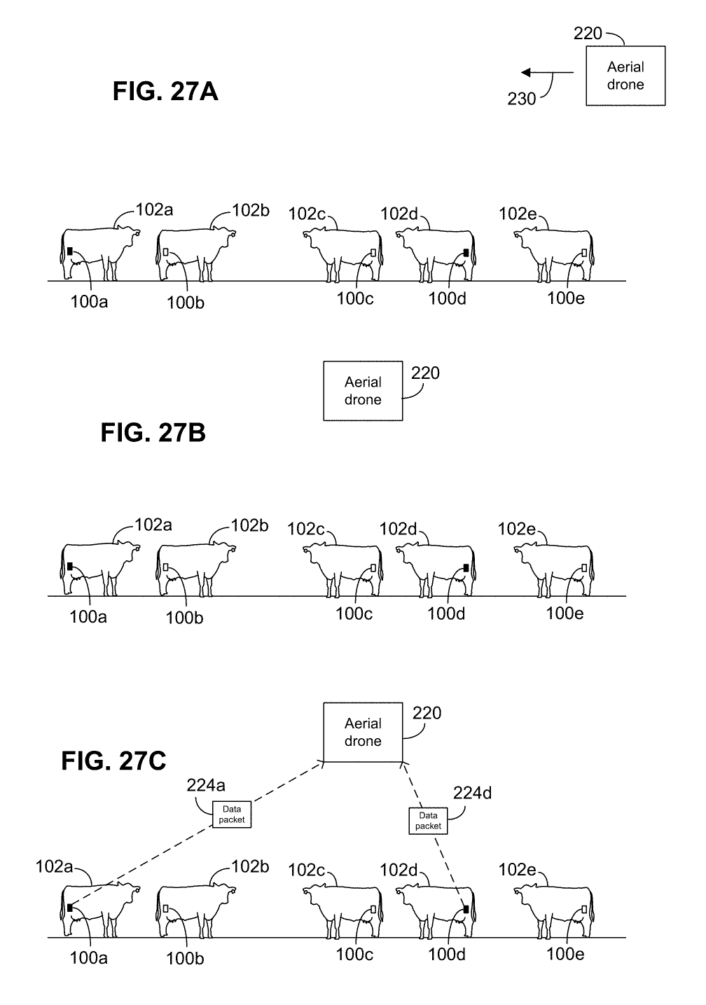

FIGS. 27A-27C show an example sequence of operation that can be implemented to perform the communication example of FIG. 25. It will be understood that a similar sequence of operation can also be implemented in the context of other communication configurations.

In FIG. 27A, multiple cows (102a-102e) are depicted as being at an area. Each cow is shown to have with it a respective patch; and similar to the example of FIG. 25, the first and fourth cows (102a, 102d) are shown to have their current fertility conditions detected by the respective patches 100a, 100d.

FIG. 27A further shows an aerial drone 220 approaching the area, as indicated by an arrow 230. In FIG. 27B, the aerial drone 220 is shown to be passing, and/or have stopped at, a location over the area. In some embodiments, such flight to, and passing over/stopping at, a given area can be based on a programmed instructions, control of a remote operator, or some combination thereof. In the example of FIG. 27B, it will be assumed that all of the cows and their respective patches are within the area covered by the aerial drone 220.

In FIG. 27C, the patches 100a, 100d of the first and fourth cows 102a, 102d are shown to transmit their respective data packets 224a, 224d, similar to the example of FIG. 25. In some embodiments, such transmission can be performed while the aerial drone is moving over the covered area, while stopped over the covered area, or some combination thereof.

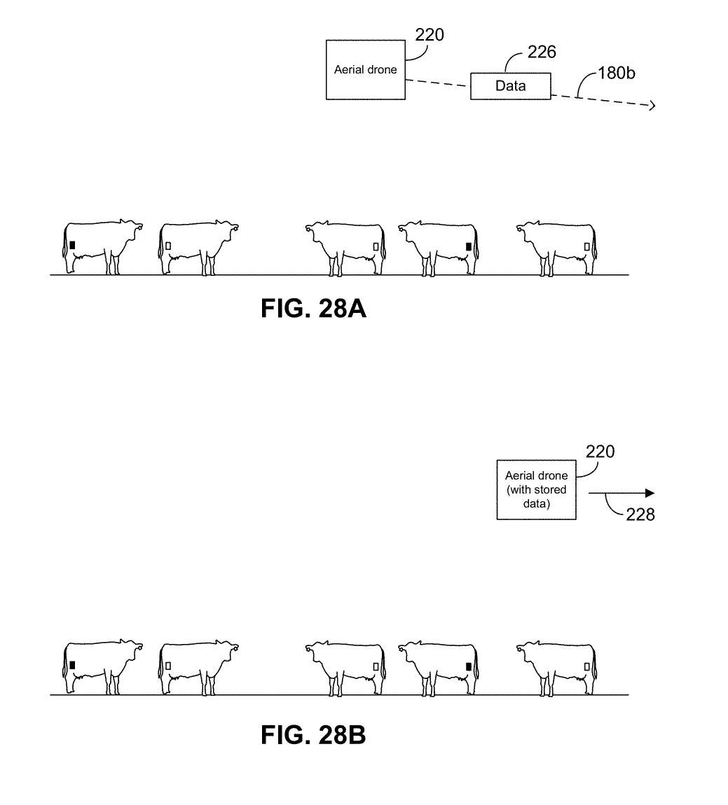

FIGS. 28A and 28B show examples of relay functionalities that can be implemented to relay the information obtained from one or more patches, such as in the example of FIGS. 27A-27C. In FIGS. 28A and 28B, it is assumed that the aerial drone 220 has received one or more data packets from respective cow(s).

FIG. 28A shows that in some embodiments, data corresponding to the data packet(s) obtained from the patch(es) can be transmitted (arrow 180b) from the aerial drone 220 to a monitor (e.g., 106 in FIG. 19). Such data sent from the aerial drone 220 can be substantially the same as the data packet(s) obtained from the patch(es), be a processed version of the data packet(s) obtained from the patch(es), etc. In some embodiments, such relaying of data by the aerial drone 220 can be performed in real time or in approximately real time, or after some delay.

FIG. 28B shows that in some embodiments, data corresponding to the data packet(s) obtained from the patch(es) can be stored in the aerial drone 220 (e.g., in a storage device). Such stored data can be carried by the aerial drone 220 as it moves (arrow 228) to a next data collection area or back to a base. Such storage of data in the aerial drone 220 may be desirable in some situations. For example, if the relay link (e.g., 180b in FIG. 28A) is not available, the data can be stored for re-transmission at a later time, and/or retrieval at the base.

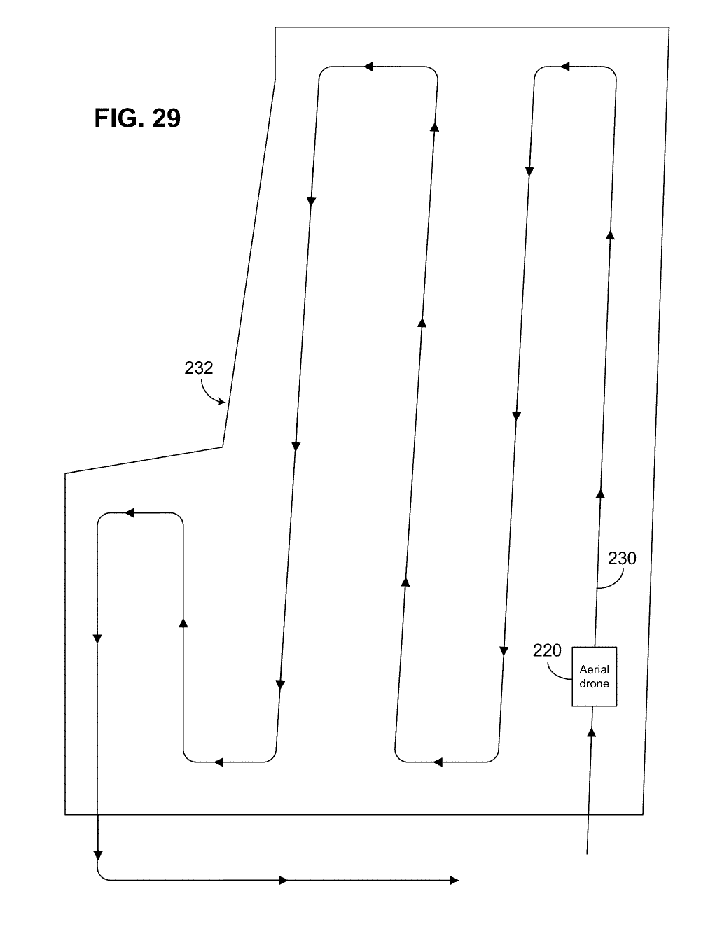

FIG. 29 shows an example application where a mobile relay device such as an aerial drone 220 can be particularly useful. Suppose that one or more groups of cows graze in a large area 232. Such a large area may have terrain that is not suitable for ground vehicles. Even if a ground vehicle is able to reach the various parts of the large area 232, there may be ground-based obstacles that inhibit or reduce the effectiveness of communication with multiple patches.

As described herein, an aerial relay platform such as an aerial drone can provide effective coverage of large areas. FIG. 29 shows that with use of an aerial drone 220 (having a relay functionality), a large area 232 can be covered in an effective manner. For example, if the locations where groups of cows are known, the aerial drone 220 can be programmed or controlled to fly to such locations and obtain data from the patches worn by the cows.

In another example, and as depicted in FIG. 29, specific locations of the cows may not be known within the large area 232. In such a situation, the aerial drone 220 can be programmed or controlled to fly in a route 230 that generally provides coverage for the large area, for obtaining data from the patches worn by the cows.

In the various examples described herein, data associated with a given patch can include identification of the cow wearing that patch. There are a number of ways such identification can be achieved. For example, suppose that the given patch has a unique identifier that generally remains fixed. When that patch is applied to the corresponding cow (having its identifier such as a tag number), such mapping of the two identifiers can be mapped into a database utilizing a portable computing device such as a tablet, a smart phone, a barcode scanner, etc. Then, when data for that patch is transmitted to a monitor, such data can include the patch identifier. Thus, identification of the cow corresponding to the data can be obtained from the database.

In another example, a given patch can be configured to allow programming of an identifier field. Such programming can be achieved, for example, when the patch is applied to a corresponding cow, with the cow's unique identifier. Then, when data for that patch is transmitted to a monitor, such data can include the cow's identifier.

Based on the foregoing patch/cow identification capability, a given cow's location can be monitored along with its fertility condition. Such location monitoring capability can provide a number of desirable features when a number of cows are being managed in a large area.

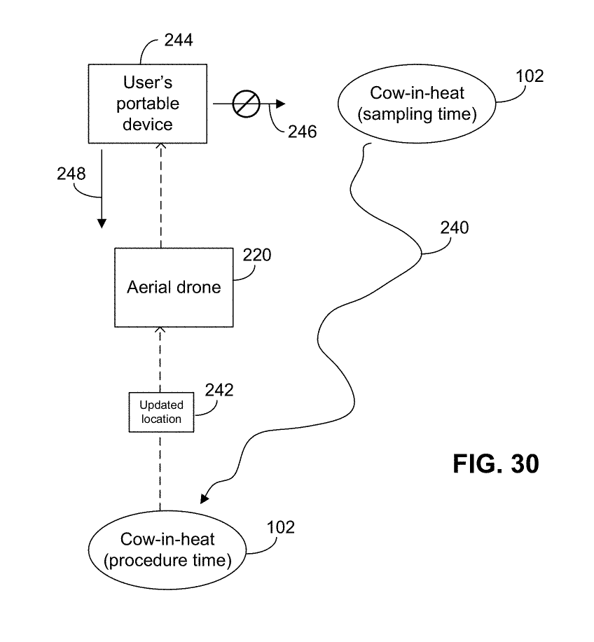

For example, and in the context of monitoring of a cow's fertility condition as described herein, FIG. 30 shows a situation where a cow's detected condition indicates that the cow (102) is in heat at the sampling time. Suppose that such detection of in-heat condition is achieved with use of an aerial drone 220. As described herein, such an in-heat condition (e.g., within or near standing estrus window) remains for a relatively short period of time. Thus, it is important for a person managing the cow to get to the cow and perform an appropriate procedure (e.g., artificial insemination or transport of the cow) to benefit from the short in-heat period.

However, and as shown in FIG. 30, suppose that subsequent to the initial detection of the cow-in-heat, the cow moves to another location (indicated as 240). In such a situation, if the person goes to the initial location based on the initial detection, that cow will not be found, and precious time will be lost.

FIG. 30 further shows that in some embodiments, a monitor (e.g., 106 in FIGS. 23 and 24) can include a portable device 244 configured to at least receive data from the aerial drone 220. With such a portable device, a user can receive updated location (242) of the cow-in-heat (102) from the same or different aerial drone 220 performing a subsequent monitoring flight. Accordingly the user can go directly to the more current location (indicated as 248), and not to the old location (indicated as 246).

It is noted that the monitoring flight of the aerial drone subsequent to the initial detection can be implemented with another routine flight pattern, or based on a more specific pattern with respect to one or more locations of cows with in-heat conditions. Either or both of such flight patterns can be achieved with a fixed frequency or at an adjusted frequency (e.g., at an increased frequency).

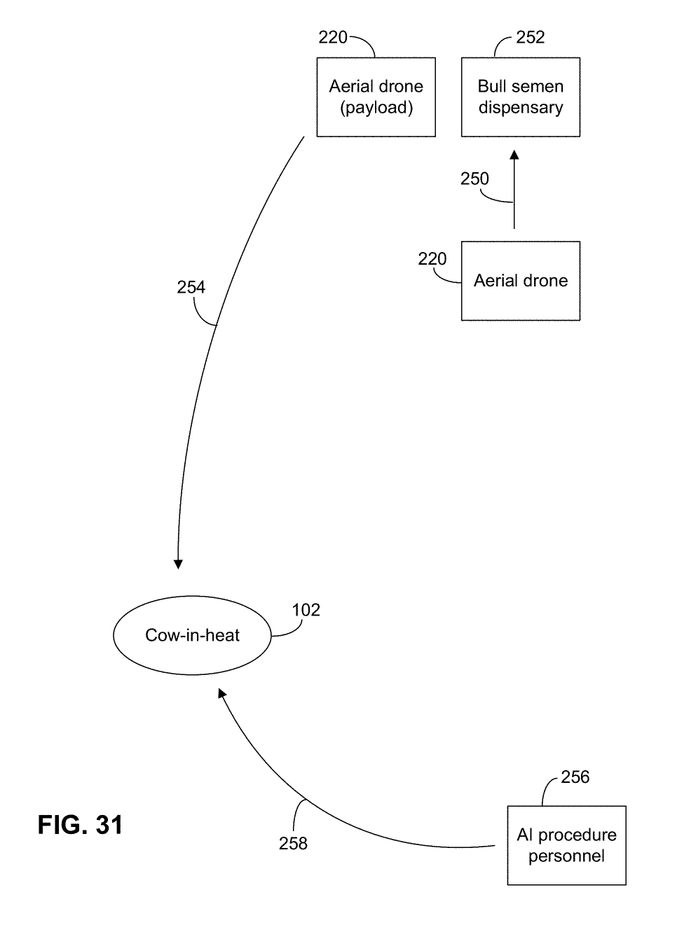

In the various examples described in reference to FIGS. 24-30, an aerial drone can be configured to facilitate various monitoring functionalities associated with one or more patches. FIG. 31 shows that in some embodiments, an aerial drone 220 can be utilized to carry a payload to facilitate fertilization of a cow-in-heat. Such an aerial drone may or may not have data relaying functionality as described herein.

In the example of FIG. 31, the aerial drone 220 can originate from, or be flown to (indicated as 250), a bull semen dispensary 252. At the dispensary 252, a bull semen in an application container can be loaded onto the aerial drone 220 as a payload. Such a payload can be flown to a cow-in-heat 102 at a detected and/or updated location. Concurrently, a person capable of performing artificial insemination (AI) can also go to the same location without having to first go to the dispensary 252. Once the bull semen is retrieved from the aerial drone, AI procedure can be performed on the cow.

In some embodiments, and as described herein, a patch can be configured to allow identification of a cow wearing the patch, and/or provide location information (e.g., a GPS coordinate information), to facilitate efficient management of cows during their fertility periods. In some embodiments, a patch having one or more features as described herein can be configured to provide time information. Such time information feature can be implemented with or without some or all of the foregoing identifier and location information.

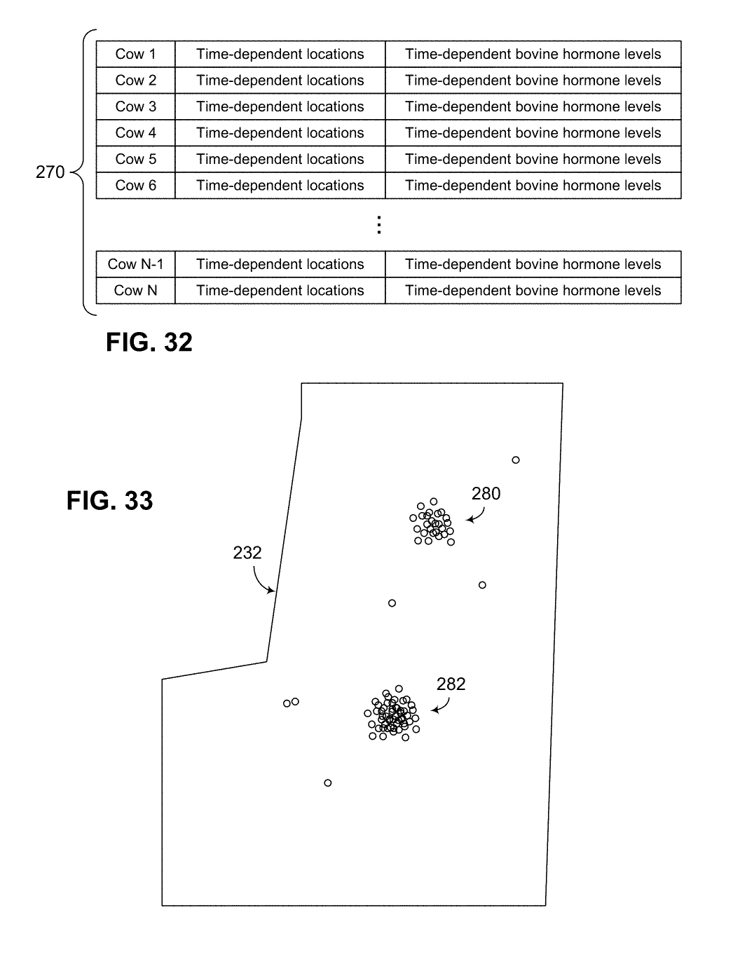

In some applications, some or all of the cow identification, location information, and time information can be utilized to provide useful information that can be beneficial for management of cows, especially if there is a large number of cows. For example, FIG. 32 shows a data set 270 obtained for N cows through use of respective patches. Each cow can be identified by the respective patch, and data for that cow can include information about one or more time-dependent locations, and one or more time-dependent hormone level measurements. As an example, suppose such time-dependent information includes time and location associated with detection of a hormone level indicative of an in-heat condition of each cow.

With a large number of samples, the data set 270 can be analyzed to provide useful information for better management of the cows. For example, FIG. 33 shows plots of locations on the example grazing area 242 where the cows enter their in-heat conditions. With such analysis, it can be easier to see a pattern revealing one or more locations where in-heat condition is more likely to occur than other locations. For example, in FIG. 33, locations 280 and 282 show clusters of data points, indicating that a cow is more likely to be in-heat at or near such locations than other locations.

Such information can be very useful for management of cows. For example, one can understand any unique features of such locations (e.g., environmental conditions) that promote higher likelihood of fertility; and such features can be replicated at other locations. In another example, such information can greatly aid in, for example, logistics associated with monitoring (e.g., flight pattern of aerial drones) and artificial insemination of the in-heat cows (e.g. make such locations more accessible).

Similarly, time information associated with the in-heat cows can be analyzed to understand or estimate when cows are likely to enter in-heat periods. With such information, cow management can be planned more efficiently. For example, if the analysis shows that cows are more likely to enter in-heat periods at certain time of a day, scheduling of personnel and/or related supplies can be adjusted accordingly.

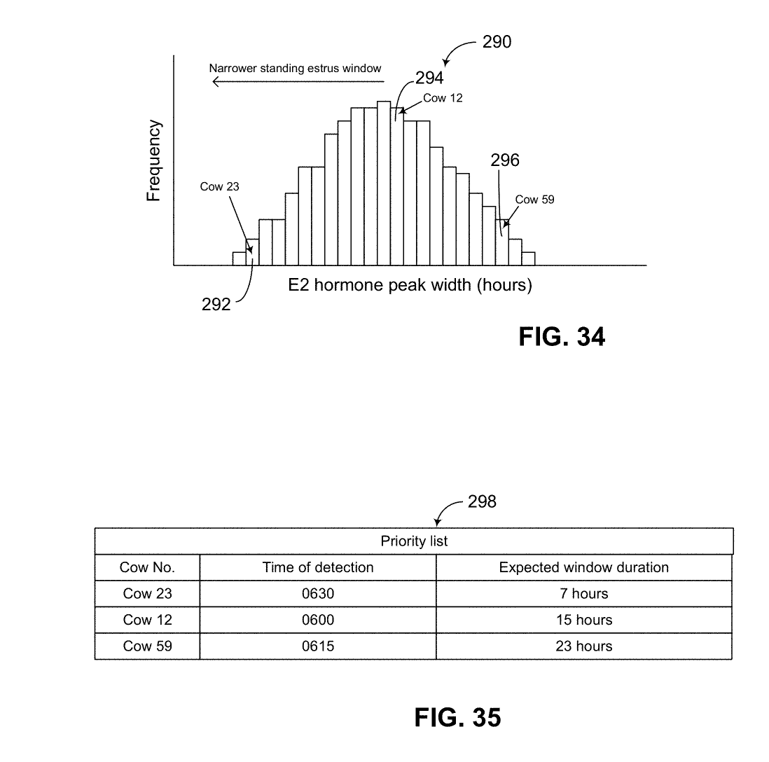

Aside from the foregoing analyses of locations and times associated with in-heat cows, more detailed measurements and related analyses can be performed to allow, for example, prioritization of tasks to be performed on such in-heat cows. For example, and referring to FIG. 34, suppose that hormone level measurements are made with patches with sufficient frequency to allow estimation of beginning and end times of estrus window of each of a group of cows.

With such information, one can readily see a distribution of standing estrus window widths; and an example of such a distribution (290) is shown in FIG. 34. For example, suppose that cows with identifiers 12, 23 and 59 are known to, or monitored to have their in-heat periods at similar times (e.g., on the same day). From the distribution of FIG. 34, one can see that Cow 23 has a narrow standing estrus window (in width bin 292), Cow 12 has an average-width standing estrus window (in width bin 294), and Cow 59 has a wide standing estrus window (in width bin 296). Thus, if such cows are to be treated on the same day, one can prioritize the treatment schedule based on such standing estrus window width.

For example, FIG. 35 shows a priority list 298 that can be generated for the example cows of FIG. 34. Assuming that such cows have similar detection times for the beginning of their respective standing estrus windows (e.g., between 0600 and 0630), the cow with the smallest window (Cow 23) can be treated first, followed by Cow 12, and then Cow 59.

In some embodiments, some or all of the foregoing analyses and listing of, for example, treatment priority can be performed automatically by a processor associated with a monitor.

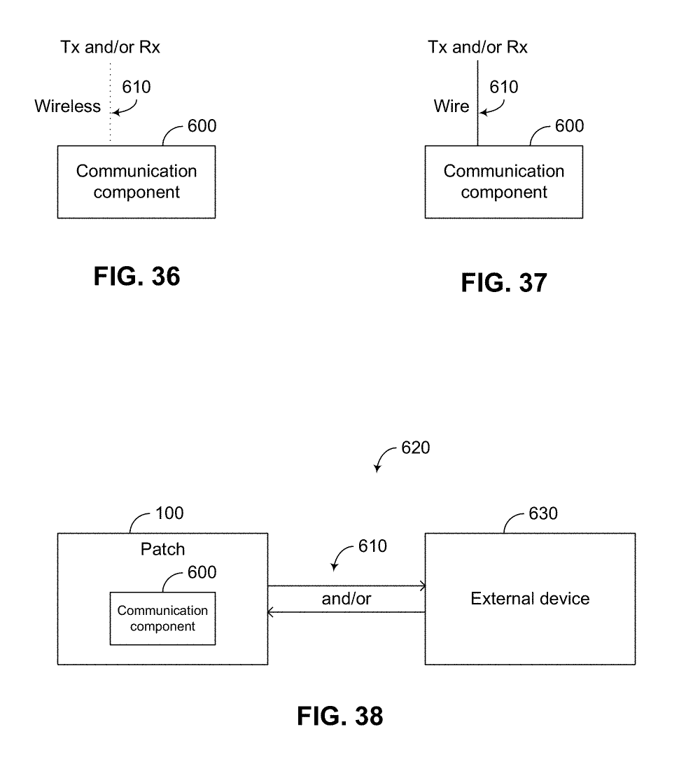

FIGS. 36-40 show examples of communications and/or system functionalities that can be implemented in a system having one or more patches as described herein. For example, FIGS. 36 and 37 show that in some embodiments, a communication component 600 (e.g., 134 in FIG. 5) of a patch can be configured to provide a wireless communication (depicted as 610 in FIG. 36) with an external device, a wired communication (depicted as 610 in FIG. 37) with an external device, or some combination thereof. For the purpose of description of FIGS. 36 and 37, an external device can be another patch, a non-patch device, etc.

In some embodiments, in each of the examples of FIGS. 36 and 37, the wireless and/or wired communication link 610 can include a transmit (Tx) functionality (relative to the corresponding patch), a receive (Rx) functionality, or any combination thereof.

FIG. 38 shows a system 620 that can be formed with one or more patches 100 as described herein, and an external device 630. For the purpose of description of FIG. 38, it will be understood that the external device 630 is relative to the patch 100. Thus, if the external device 630 is another patch, then the patch 100 shown in FIG. 38 can be considered to be external to the other patch (630). As described in reference to FIGS. 36 and 37, it will be understood that the external device 630 can be a patch that may or may not be similar to the patch 100.

In the example of FIG. 38, the patch 100 is shown to include a communication component similar to the examples of FIGS. 36 and 37. Accordingly, the communication between the patch 100 and the external device 630 can include transmit and/or receive portions.

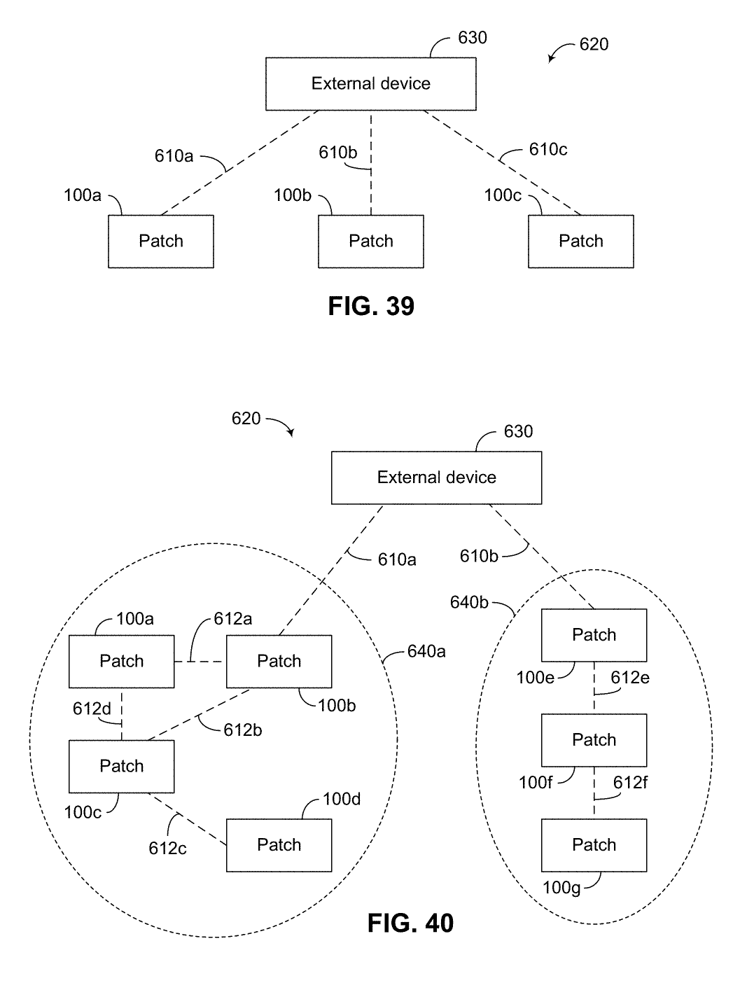

FIG. 39 shows that in some embodiments, the system 620 of FIG. 38 can include a plurality of patches 100 that communicate with a common external device. For example, a system 620 of FIG. 39 is shown to include a plurality of patches 100a, 100b, 100c and an external device 630. More particularly, the first patch 100a can be in communication (610a) with the external device 630, the second patch 100b can be in communication (610b) with the external device 630, and the third patch 100c can be in communication (610c) with the external device 630. In some embodiments, such an external device can be configured to, for example, coordinate operations of the patches (100a, 100b, 100c), collect data from the patches, etc. In some embodiments, the external device 630 can be configured to communicate with another device at a similar level, with another device at a higher level, or any combination thereof.

FIG. 40 shows that in some embodiments, the system 620 of FIG. 38 can include a plurality of patches 100 that can communicate with each other, and/or with an external device. For example, a first group (640a) of patches and a second group (640b) are shown to be included in a system 620, and generally in communication with an external device 630. More particularly, the first group 640a is shown to include four example patches 100a, 100b, 100c, 100d, and the second group 640b is shown to include three example patches 100e, 100f, 100g. Such first and second groups 640a, 640b of patches can be grouped based on, for example, physical proximity/separation, different functionalities, etc.

In some embodiments, within a given group, each of the plurality of patches can communicate directly with the external device 630, through a representative patch, or some combination thereon. For example, for the first group 640a, the patches 100a and 100b are shown to have a communication link 612a; the patches 100a and 100c are shown to have a communication link 612d; the patches 100c and 100d are shown to have a communication link 612c; and the patches 100c and 100b are shown to have a communication link 612b. Further, the patch 100b is shown to be a representative communication member and be in communication (610a) with the external device 630.

In another example, for the second group 640b, the patches 100e and 100f are shown to have a communication link 612e; and the patches 100f and 100g are shown to have a communication link 612f. Further, the patch 100e is shown to be a representative communication member and be in communication (610b) with the external device 630.

In some embodiments, the communication links between the patches within a given group can be based on, for example, relative proximity/distance among the users wearing the respective patches, some hierarchy of the users and/or patches, or some combination thereof. In some embodiments, the communication links between the patches can be configured as a mesh network, or be based on such a network.

In some embodiments, a system of patches as described herein (e.g., in reference to FIGS. 36-40) can provide a system level information that may not be available from an individual patch.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein may be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. A patch comprising:
a patch substrate configured to support a plurality of components, and to allow the patch to be attached to a cow;
a detector implemented on or at least partially within the patch structure, and configured to detect one or more conditions associated with a fertility cycle of the cow; and
an interface component implemented on or at least partially within the patch structure, and in communication with the detector, the interface component configured to transmit information related to the one or more conditions, such that the information allows estimation of occurrence of a standing estrus window for the cow.

2. The patch of claim 1 wherein the patch substrate is configured to be attached to a skin of the cow.

3. The patch of claim 1 wherein the detector is configured to detect presence and/or concentration of a hormone associated with the fertility cycle of the cow.

4. The patch of claim 3 wherein the detector is configured to measure a concentration level of progesterone hormone.

5. The patch of claim 4 wherein the detector is further configured to generate a signal indicative of an approach of the standing estrus window when the concentration levels measured at two different times indicate a decrease from a first value above a threshold to a second value below the threshold.

6. The patch of claim 3 wherein the detector is configured to measure a concentration level of estradiol hormone.

7. The patch of claim 6 wherein the detector is further configured to generate a signal indicative of a beginning of the standing estrus window when the concentration levels measured at two different times indicate an increase from a first value below a threshold to a second value above the threshold.

8. The patch of claim 3 wherein the detector is configured to measure a concentration level of luteinizing hormone.

9. The patch of claim 8 wherein the detector is further configured to generate a signal indicative of a beginning of the standing estrus window when the concentration levels measured at two different times indicate an increase from a first value below a threshold to a second value above the threshold.

10. The patch of claim 1 wherein the detector is configured to detect a rate of change in concentration of a hormone associated with the fertility cycle of the cow.

11. The patch of claim 1 wherein the detector is configured to detect an activity of the cow, the activity associated with the fertility cycle of the cow.

12. The patch of claim 11 wherein the activity includes an increase in number of steps taken by the cow within a given time interval.

13. The patch of claim 12 wherein the detector includes a pedometer functionality.

14. The patch of claim 11 wherein the activity includes a mounting action associated with the cow.

15. (canceled)

16. (canceled)

17. The patch of claim 11 wherein the activity includes the cow being in close proximity of another cow.

18. The patch of claim 17 wherein the detector includes a proximity sensing functionality with respect to a patch attached to the other cow.

19. The patch of claim 1 wherein the interface component includes a communication circuit configured to send the information to an external device.

20. The patch of claim 19 wherein the communication circuit is configured to send the information in a wireless manner.

21. A method comprising:
attaching a patch to a cow, the patch including a substrate configured to support a plurality of components;
detecting, with the patch, one or more conditions associated with a fertility cycle of the cow; and
transferring information about the detecting such that the information allows estimation of occurrence of a standing estrus window for the cow.

22. A system for managing fertility of cows, comprising:
a patch configured to be attached to a cow, the patch including a detector configured to detect one or more conditions associated with a fertility cycle of the cow, the patch further including an interface component in communication with the detector, and configured to transmit information related to the one or more conditions; and
a monitor configured to receive the information from the patch, such that the information received by the monitor allows estimation of occurrence of a standing estrus window for the cow.

23-31. (canceled)
